(19) United States
(12) Patent Application Publication
(10) Pub. No.: US 2024/0368667 A1
(43) Pub. Date: Nov. 7, 2024

(54) RAPID, LABEL-FREE ANTIBIOTIC SUSCEPTIBILITY OF BACTERIA DIRECTLY FROM POSITIVE BLOOD OR BODILY FLUID/CULTURE

(71) Applicant: GEORGIA TECH RESEARCH CORPORATION, Atlanta, GA (US)

(72) Inventors: Robert Martin DICKSON, Atlanta, GA (US); Alexandra B. FILBRUN, Boston, MA (US)

(21) Appl. No.: 18/292,738

(22) PCT Filed: Jul. 26, 2022
(86) PCT No.: PCT/US2022/038372

§ 371 Date: Jan. 26, 2024

Related U.S. Application Data
(60) Provisional application No. 63/225,739, filed on Jul. 26, 2021.

(51) Int. Cl. C12Q 1/24; C12Q 1/18

(52) U.S. Cl. CPC C12Q 1/24; C12Q 1/18

(57) ABSTRACT

This invention relates to rapid, novel, high-efficiency bacterial recovery systems, kits, and methods that remove essentially all blood background, while maintaining bacterial viability. This approach couples selective hemolysis with centrifugation through a separating material for rapid recovery of near-pure bacteria with minimal sample handling.

### CROSS-REFERENCE TO RELATED APPLICATIONS

This application claims benefit of U.S. Provisional Application No. 63/225,739, filed Jul. 26, 2021, incorporated herein by reference in its entirety.

### BACKGROUND

In large part arising from rapid development of antimicrobial resistance (AMR), bacterial infections remain a major cause of mortality and morbidity.¹⁻⁴ The US Centers for Disease Control and Prevention (CDC) estimates that more than 2.8 million antibiotic-resistant infections occur within the United States (U.S.) each year resulting in more than 35,000 deaths.⁵ A recent study found that the incidence of antibiotic-resistant infections more than doubled from 700,000 in 2002 to 1.6 million in 2014.6 If left unchallenged, the annual worldwide death toll attributed to antimicrobial resistance is predicted to reach 50 million by 2050.7 Particularly worrisome is the increased prevalence of AMR among bacteremia and sepsis patients.^{6, 8, 9} The exceedingly low bacterial burdens causing bacteremia and sepsis, coupled with the exceedingly high mammalian cell backgrounds, arguably make these conditions the most challenging of all bacterial infections to diagnose rapidly. As such, initial treatment often relies on the administration of empiric, broad-spectrum antibiotics that may be ineffective on (multi) drug resistant strains and can threaten the efficacy of already limited treatment options. 10-13 Consequently, shortening the time to targeted antibiotic treatment is crucial in promoting positive bacteremia and sepsis patient outcomes and stewardship of antimicrobials. Because infecting bacteria are present at 1-100 colony-forming-units (CFU) per mL blood (10⁷-10⁹-fold lower concentrations than mammalian blood cells), blood culturing remains a crucial first step in bacteremia (sepsis) diagnosis, despite its multiday delay in identifying targeted treatments. Moreover, the need for pure bacterial cultures for downstream identification and antibiotic susceptibility testing (AST) further impedes early administration of appropriate treatment. Because blood culturing remains an indispensable first step in clinical diagnosis, development of a rapid, facile, direct-from-positive blood culture AST is an important goal.

Several direct-from-positive blood culture AST workflows have been proposed for Vitek-2 (bioMérieux Inc., Durham, NC) and Phoenix (BD, Franklin Lakes, NJ) AST systems based on bacterial recovery through differential centrifugation¹⁴⁻¹⁶ or serum separation tubes.¹⁶⁻¹⁹ Although sufficient separation was reported for direct susceptibility determinations, differential centrifugation-based methods experience increased protocol complexity and user intervention through incorporation of multiple centrifugation steps and have demonstrated the need for increased culture volumes for gram positive cocci¹⁴—thus requiring knowledge of gram status prior to separation.

Separation protocols involving serum separation tubes relied on either the incorporation of a 15-minute saponin hemolysis step prior to centrifugation^{17, 19} that increased processing times and complexity, or manual harvesting of bacterial cells from the top of the gel layer with a swab. Further, bacterial recovery from the thixotropic gel in serum separation tubes generates a strong scattered light signal preventing its use within flow cytometry-based ASTs. While Walsh, et. al. coupled selective blood cell lysis and density centrifugation for direct identification of bacteria in blood cultures with high accuracy,²⁰ bacterial pellets were analyzed for identity directly within the recovery device. Therefore, additional considerations (and steps) are needed to ensure clean recovery and viability of bacteria for downstream antibiotic susceptibility testing. Instead of using the thixotropic gel in serum separation tubes, centrifugation with complex sucrose-based density cushions has shown promise in the recovery of bacteria from complex matrices (e.g. soil, blood, and nanomaterials).²¹⁻²⁴ While this approach may offer a cost-effective option for clinical laboratories, direct recovery of bacteria from the blood in this system,^{21, 22} leads to incomplete blood product rejection, scattering background, and further downstream analysis challenges unless additional subculturing-based purification steps are incorporated. Further, this incomplete mammalian cell product rejection precludes use of fast flow cytometric-based ASTs.

Flow cytometry has demonstrated promise in the rapid assessment of bacterial antibiotic susceptibilities.²⁵⁻³² One such approach, FASTinov (Porto, Portugal), classifies bacteria as sensitive, intermediate, or resistant by monitoring antibiotic-induced changes in flow cytometric fluorescence signals following 1-hour incubation of antibiotics and dye with bacteria.²⁶ While promising and commercially available, this approach does not produce minimum inhibitory concentrations (MICs) and relies on the somewhat qualitative uptake of dye by antibiotic-stressed bacteria. Potentially similar to reactive oxygen species (ROS) generation coupled with dye uptake upon antibiotic stress, this may be highly strain and antibiotic pair dependent.

Avoiding unpredictable dye-bacteria interactions, a rapid, label-free approach based on antibiotic-induced changes in bacterial morphology encoded in scatter signatures of treated and untreated bacteria was developed.³¹ Although statistically relevant shifts in scatter patterns at near-MIC antibiotic exposure were readily observed after only 1 hour, this approach was only demonstrated on gram-negative infections. Broeren et. al. demonstrated the utility of antibiotic-induced changes in flow cytometric count rates for susceptibility determinations within 90 minutes on pure bacterial populations,²⁸ but obtaining pure bacteria from blood cultures usually requires multiple slow subculturing steps.

Antimicrobial susceptibility tests (ASTs) are performed on blood and urine samples of patients when admitted to the hospital. This determines if bacteria are present which could be the cause of their illness, and potentially lead to sepsis (very serious). There are a large number of samples that need to be processed by the clinical microbiology labs in hospitals as bacteremia and sepsis are one of the top 10 causes of hospital deaths, and antimicrobial resistance proliferation is increasing, making treatment even more difficult. Faster processing of samples and identification of appropriate treatment requires faster ASTs to be developed, as slow ASTs are currently the limiting step.

What is needed in the art is the ability to quickly recover enriched samples of bacteria directly from biological samples.

### SUMMARY

Disclosed herein is a method of obtaining an enriched population of bacterial cells from a biological sample or culture, the method comprising the steps of: providing a tube comprising a separating material, wherein a proximal end of the tube is comprised of a stopper which allows entry of a needle, and wherein the separating material is in the proximal end of the tube so that the separating material is in contact with the proximal end of the tube; inserting the biological sample into the tube, wherein the biological sample is layered on the distal side of the separating material, so that the separating material is not in contact with the proximal end of the tube; centrifuging the tube for a sufficient time to cause bacterial cells to migrate into the separating material, wherein 50% or more of mammalian cells are excluded from the separating material; and withdrawing a sample from the separating material, wherein said sample comprises an enriched population of biological cells.

Further disclosed is a tube for separating bacteria from mammalian cells, wherein the tube comprises a separating material, wherein a proximal end of the tube is comprised of a material which allows entry of a needle, and wherein the separating material is in the proximal end of the tube so that the separating material is in contact with the proximal end of the tube.

Also disclosed is a kit for obtaining a bacterial sample, the kit comprising a tube for separating bacteria from mammalian cells, wherein the tube comprises a separating material, wherein a proximal end of the tube is comprised of a material which allows entry of a needle, and wherein the separating material is in the proximal end of the tube so that the separating material is in contact with the proximal end of the tube.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1A-D shows a schematic of sample loading. Positive blood culture media (a1) and culture lysate (a2) is collected with the stopcock closed to the perpendicular port. Prior to removal from the blood culture or centrifuge tube, the stopcock is closed to the needle. b. The needle is inserted into the inverted separator tube, the blood culture is injected above the sucrose cushion, and the upper air is evacuated. c. The stopcock is closed to the syringe and the needle removed from the tube. d. The separator tube containing sample and used loading apparatus is shown.

FIG. 2A-D shows photographs of inverted recovery tubes containing S. aureus blood culture without (a & b) and with (c & d) saponin pre-treatment. (a & c) recovery tubes before centrifugation; (b & d) recovery tubes following a 10-minute centrifugation at 3,500×g. After centrifugation, near-pure bacteria are present in the sucrose layer (bracket) and collected by inserting a needle through the septum (pink plug at bottom of tube)

FIG. 3A-D shows (A) Antibiotic-induced flow cytometric scatter signals of F. coli Mu76 treated with ceftazidime. Sucrose-recovered E. coli Mu76 was treated with ceftazidime for 4 hours (37° C.) and analyzed with high-throughput flow cytometry. (B) Total bacterial scatter count rate vs antibiotic concentration. (C) Overlap between no-antibiotic control in (A) and 1-standard deviation contours for each antibiotic exposed sample. (D) Combination of B and C to give count rates adjusted for differences in scatter position of untreated and antibiotic-treated bacteria. MIC as determined by broth microdilution was 2 μg/mL.

FIG. 4 shows support vector machine (SVM) classifier as found by grid search and cross-validation with Baclight dye-stained bacterial cell samples. Gram positive pathogens (blue markers) appear at higher red fluorescence due to greater hexidium iodide (HI) uptake. The weighted average precision and sensitivity rates for the optimized SVM model are 94%. Each dot corresponds to an individual bacterial detection, with 0.2% of data reported for each isolate; individual replicates each of three Gram positive and seven Gram negative bacteria in total were evaluated. EC, Mu890: Escherichia coli; KP: Klebsiella pneumoniae, PA: Pseudomonas aeruginosa; MSSA, MRSA, SA: Staphylococcus aureus.

FIG. 5A-D shows flow cytometric scatter patterns of (a) PBS, (b) CAMHB, (c) 55% sucrose, and (d) sucrose gel. FSC: forward scatter; SSC (side scatter).

FIG. 6A-C shows effects of sucrose on bacterial growth of (a) E. coli, (b) P. aeruginosa, and (c) S. aureus. Sucrose recovery solutions were diluted various amounts in media and growth monitored at 37° C. with shaking via OD-600 measurements. Overnight cultures of bacteria recovered through plating were also inoculated into media and used as a control.

FIG. 7A-C shows growth of 10× recovery solutions against bacteria at different stages along AST workflow. Growth of 10-fold diluted recovery solutions, dilute blood culture media, colony resuspension, and sub-cultures was monitored at 37° C. with shaking via OD-600 measurements. (a) E. coli; (b) P. aeruginosa; (c) S. aureus.

FIG. 8 shows susceptibility determination of E. coli Mu76 treated with ceftazidime. Sucrose-recovered E. coli Mu76 was treated with ceftazidime for 4 hours (37° C.) and analyzed with high-throughput flow cytometry. Modified count rate ratios, accounting for scatter position, as a function of log 2 antibiotic concentration were recursively fit to a 3 point linear function with a frameshift of n+1 between iterations. Trend lines consisting of slopes<−0.01 represent resistance (i.e. bacterial signal continues to descend with increased antibiotic concentration), while slopes≥−0.01 represent susceptibility. The MIC was set as the lowest antibiotic concentration that resulted in the maintenance of a slope≥−0.01 for all subsequent 3-point linear fits (1 μg/mL).

FIG. 9A-D shows antibiotic-induced cytometric changes. Flow cytometric (A) count rate and (B) scatter position overlap of P. aeruginosa 7 treated with meropenem. Sucrose-recovered P. aeruginosa 7 was treated with meropenem for 4 hours (37° C.) and analyzed with high-throughput flow cytometry. (C) Count rates adjusted for differences in scatter position of untreated and antibiotic-treated bacteria. (D) Observed growth (determined by plating and reported as the percentage of colonies relative to those in the no-antibiotic control) for antibiotic concentrations near the transition point, where increased raw counts were observed (panel A). MIC as determined by broth microdilution was 1 μg/mL. MIC as determined by our overlap-modified count rate (C) is 2 μg/mL, as further quantified in FIG. 10.

FIG. 10A-H shows susceptibility determination of P. aeruginosa 7 treated with meropenem. Sucrose-recovered P. aeruginosa 7 was treated with meropenem for 4 hours (37° C.) and analyzed with high-throughput flow cytometry. Overlap-modified count rate ratios, accounting for scatter position, as a function of log base 2 antibiotic concentration were recursively fit to a 3 point linear function with a frameshift of n+1 between iterations. Trend lines consisting of slopes<−0.01 (C, D, E) represent resistance (i.e. bacterial signal continues to descend with increased antibiotic concentration), while slopes≥−0.01 (A, B, F, G, H) represent susceptibility. The MIC was set as the lowest antibiotic concentration that resulted in the maintenance of a slope≥−0.01 for all subsequent 3-point linear fits. This gives the MIC of 2 μg/mL for meropenem.

FIG. 11 shows a schematic of the direct-from-positive blood culture AST timeline.

FIG. 12A-B shows a schematic of (A) a tube which can be used for obtaining an enriched population of bacterial cells from a biological sample; and (B) the tube with a biological sample being inserted above the layer of separating material.

FIG. 13A-C shows antibiotic-induced cytometric changes. Flow cytometric (A) count rate and (B) scatter position overlap of P. aeruginosa treated with meropenem. Sucrose-recovered P. aeruginosa was treated with meropenem for 7 hours (37° C.) and analyzed with high-throughput flow cytometry. (C) Count rates adjusted for differences in scatter position of untreated and antibiotic-treated bacteria. MIC as determined by broth microdilution was 1 μg/mL.

FIG. 14A-B shows antibiotic-induced flow cytometric changes of E. coli treated with tobramycin. (A) Scatter patterns and (B) count rates both change with increased antibiotic concentration. At concentrations≥0.25 μg/mL (MIC as determined by BMD), the count rate is significantly reduced (≥˜100 evts/s) when compared to the no-antibiotic control (˜11,000 evts/s). As the inoculum source is consistent across samples, the low count rate observed for samples treated with ≥MIC antibiotic concentrations suggests background contributes little to overall observed signals. FSC: forward scatter; SSC: side scatter.

FIG. 15A-D shows scatter plots comparing negative and positive blood cultures. Negative (left panels, A and C) and positive (right panels B and D) blood cultures from a single colony of E. coli strain Mu890 spiked in human blood at 0-(top row, A and B) and 4-hour (bottom row, C and D) incubation (37° C.) after recovery through selective hemolysis and centrifugation through inverted tubes containing sucrose. Solutions recovered through sucrose were diluted to mimic experimental conditions. Dilutions for positive and negative cultures were identical. Count rates are indicated as numerical values within each plot at 12 mL/min flow rates.

FIG. 16 shows bar chart of count rates from negative (neg) and positive (pos) blood cultures presented in FIG. 15. Inset: expansion of the y-axis to show the very low counts/see for positive cultures at 0 hours and negative cultures at both 0 hour and 4 hour incubation (37° C.) time points.

### DETAILED DESCRIPTION

### Definitions

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g., rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5).

Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The term “organism,” “subject,” or “host” refers to any living entity, including humans, mammals (e.g., cats, dogs, horses, mice, rats, pigs, hogs, cows, and other cattle), birds (e.g., chickens), and other living species that are in need of treatment. In particular, the term “host” includes humans. As used herein, the term “human host” or “human subject” is generally used to refer to human hosts. In the present disclosure the term “host” typically refers to a human host, so when used alone in the present disclosure, the word “host” refers to a human host unless the context clearly indicates the intent to indicate a non-human host.

The term “microorganism” or “microbe” as used herein refers to a small (often, but not always, microscopic) organism that is typically, but not exclusively, single cellular, and includes organisms from the kingdoms bacteria, archaea, protozoa, and fungi. The present disclosure is primarily directed to microorganisms that are pathogenic and capable of causing disease. In embodiments, microorganism includes bacteria and fungi capable of causing disease, particularly disease in humans and other mammals and animals in need of treatment.

The term “sample” can refer to a tissue sample, cell sample, a fluid sample, and the like. A sample may be taken from a host. The tissue sample can include hair (including roots), buccal swabs, blood, saliva, semen, muscle, or from any internal organs. The fluid may be, but is not limited to, urine, blood, ascites, pleural fluid, spinal fluid, semen, wound exudates, sputum, fecal matter, saliva, and the like. The body tissue can include, but is not limited to, skin, muscle, endometrial, uterine, and cervical tissue. While a sample, in the context of the present disclosure, is primarily a biological sample (e.g., from a living host) the sample may also be an environmental sample suspected of contamination by microbes, such as a water sample, food sample, soil sample, and the like. Although a liquid sample and some solid samples may be used as a test sample without modification for testing directly, if a solid sample is to be made into liquid form for testing and/or a liquid sample is to be diluted, a test sample may be made by reconstituting, dissolving, or diluting the sample in a fluid such as water, buffered saline, and the like.

The term “blood” as used herein means either whole blood or any one, two, three, four, five, six, or seven cell types from the group of cell types consisting of red blood cells, platelets, neutrophils, eosinophils, basophils, lymphocytes, and monocytes. Blood can be from any species including, but not limited to, humans, any laboratory animal (e.g., rat, mouse, dog, chimp), or any mammal.

The term “blood culture” as used herein refers to any amount of blood that has been mixed with blood culture media. Examples of culture media include, but are not limited to, supplemented soybean casein broth, soybean casein digest, hemin, menadione, sodium bicarbonate, sodium polyaneltholesulfonate, sucrose, pyridoxal HCKI, yeast extract, and L-cysteine. One or more reagents that may be used as blood culture media are found, for example, in Stanier et al., 1986, The Microbial World, 5th edition, Prentice-Hall, Englewood Cliffs, N.J., pages 10-20, 33-37, and 190-195, which is hereby incorporated by reference herein in its entirety for such purpose. In some instances, a blood culture is obtained when a subject has symptoms of a blood infection or bacteremia.

The term “culture” as used herein refers to any biological sample from a subject that is either in isolation or mixed with one or more reagents that are designed to culture cells. The biological sample from the subject can be, for example, blood, cells, a cellular extract, cerebral spinal fluid, plasma, serum, saliva, sputum, a tissue specimen, a tissue biopsy, urine, a wound secretion, a sample from an in-dwelling line catheter surface, or a stool specimen. The subject can be a member of any species including, but not limited to, humans, any laboratory animal (e.g., rat, mouse, dog, chimp), or any mammal. One or more reagents that may be mixed with the biological sample are found, for example, in Stanier et al., 1986, The Microbial World, 5th edition, Prentice-Hall, Englewood Cliffs, N.J., pages 10-20, 33-37, and 190-195, which is hereby incorporated by reference herein in its entirety for such purpose. A blood culture is an example of a culture. In some embodiments, the biological sample is in liquid form and the amount of the biological sample in the culture is between 0.1 ml and 150 ml, between 2 ml and 100 ml, between 0.5 ml and 90 ml, between 0.5 ml and 10,000 ml, or between 0.25 ml and 100,000 ml. In some embodiments, the biological sample is in liquid form and is between 1 and 99 percent of the volume of the culture, between 5 and 80 percent of the volume of the culture, between 10 and 75 percent of the volume of the culture, less than 80 percent of the volume of the culture, or greater than 10 percent of the volume of the culture. In some embodiments, the biological sample is between 1 and 99 percent of the total weight of the culture, between 5 and 80 percent of the total weight of the culture, between 10 and 75 percent of the total weight of the culture, less than 80 percent of the total weight of the culture, or greater than 10 percent of the total weight of the culture.

### General Description

Being able to separate out microbes from biological samples in a timely manner is critically important. Detection, culturing, and typing these microbes can allow for a subject to be appropriately treated. Disclosed herein are tubes and kits, as well as methods of obtaining an enriched population of bacterial cells.

Specifically, disclosed herein is a method of obtaining an enriched population of bacterial cells from a biological sample, the method comprising the steps of: providing a tube (1) comprising a separating material (2), wherein a proximal end (3) of the tube is comprised of a stopper (4) which allows entry of a needle (5), and wherein the separating material is in the proximal end of the tube so that the separating material is in contact with the proximal end of the tube; inserting the biological sample (6) into the tube, wherein the biological sample is layered on a distal side (7) of the separating material, so that the separating material is not in contact with the proximal end of the tube; centrifuging the tube for a sufficient time to cause bacterial cells to migrate into the separating material, wherein 50% or more of mammalian cells are excluded from the separating material; and withdrawing a sample from the separating material, wherein said sample comprises an enriched population of biological cells.

By “enriched population of bacterial cells” is meant that the concentration of bacterial cells in the final sample (after separation and removal), as compared to the starting, or biological sample, is increased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any amount above, below, or in-between these amounts. In one embodiment, the concentration of bacterial cells in the final sample is increased by 50% or more as compared to the starting, or biological sample.

As stated above, mammalian cells are largely excluded from the separating material during centrifugation, while bacterial cells migrate into the separating material. This process causes the enrichment of bacterial cells in the separating material. This is because bacterial cells separate out during centrifugation at a different rate based on their density. This difference can be caused by lysis of mammalian cells, which is explained in more detail below. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of mammalian cells can be excluded from the separating material after centrifugation, as compared to the amount in the starting, or biological, sample. Put another way, the concentration of mammalian cells in the final sample can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% less than the concentration of mammalian cells in the starting, or biological sample. Thus, the concentration of bacteria in the final sample can comprise at least 10, 101, 102, 103, 104, 105, 106, 107, 108 or 109 CFU/ml, preferably 102-108, 103-107 or 104-106 CFU/ml.

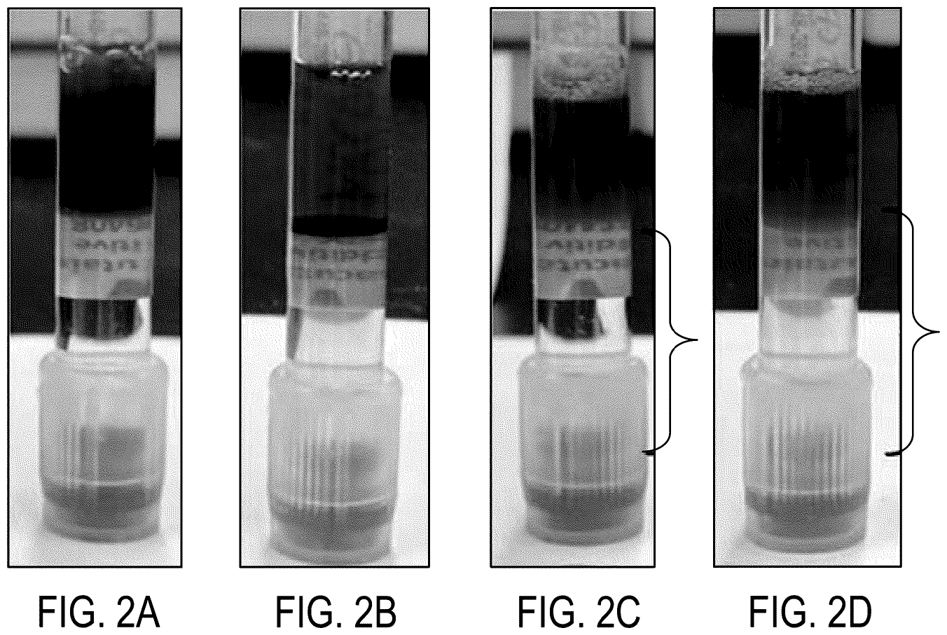

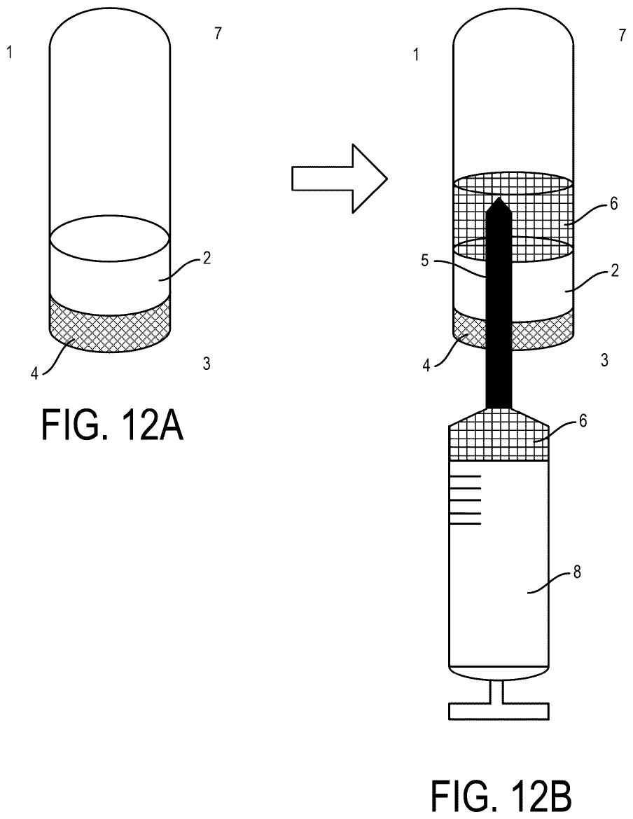

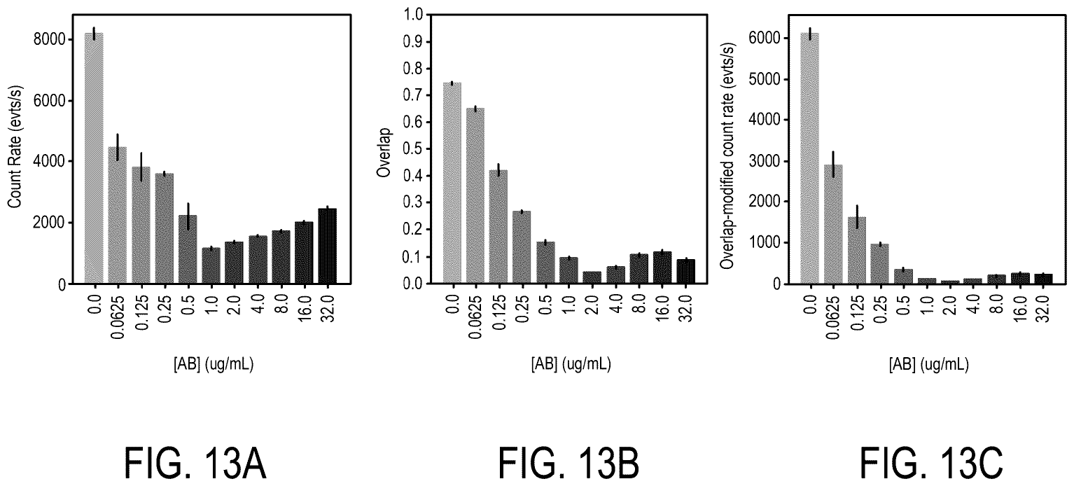

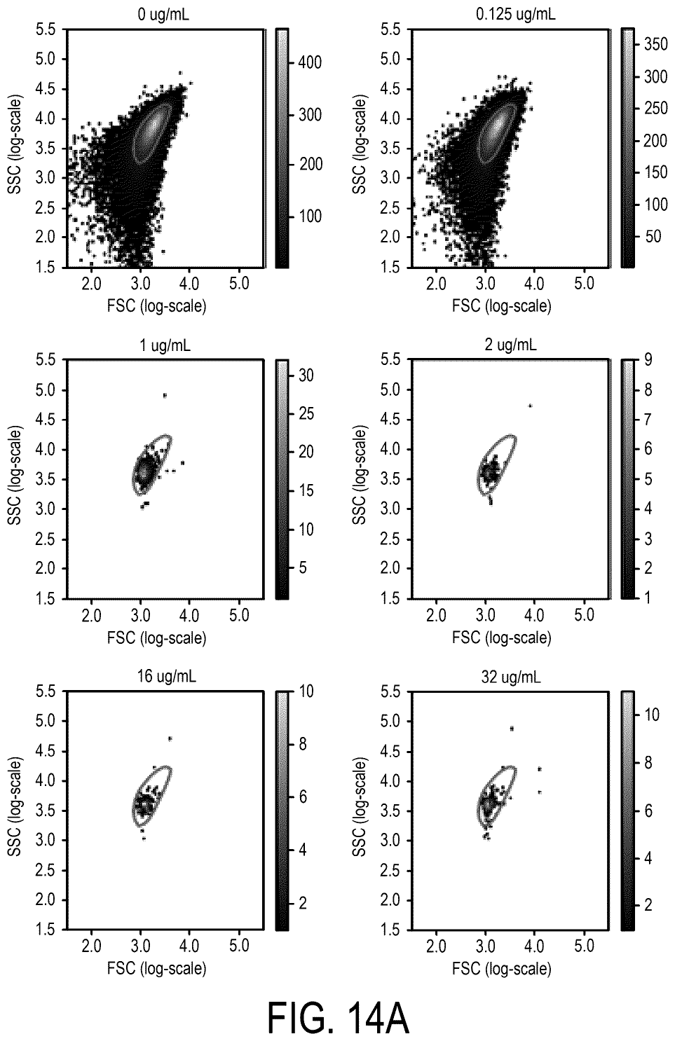

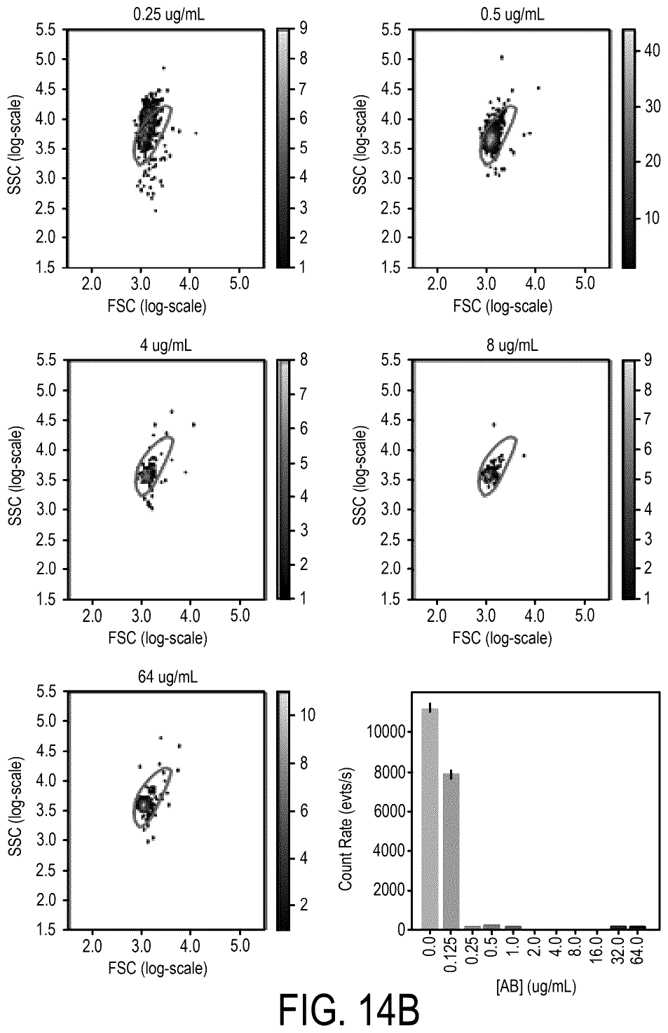

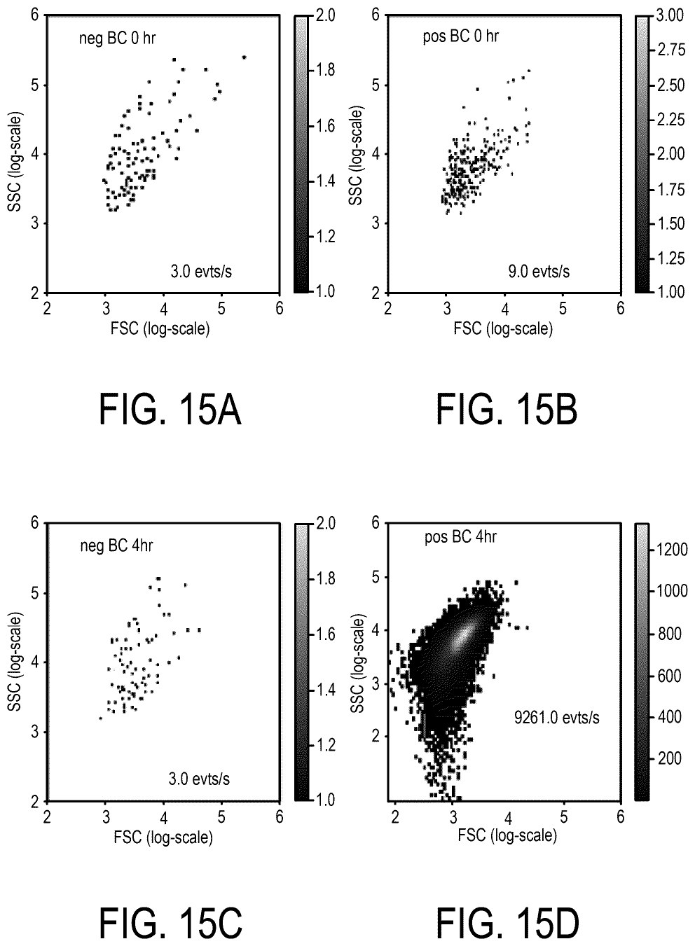

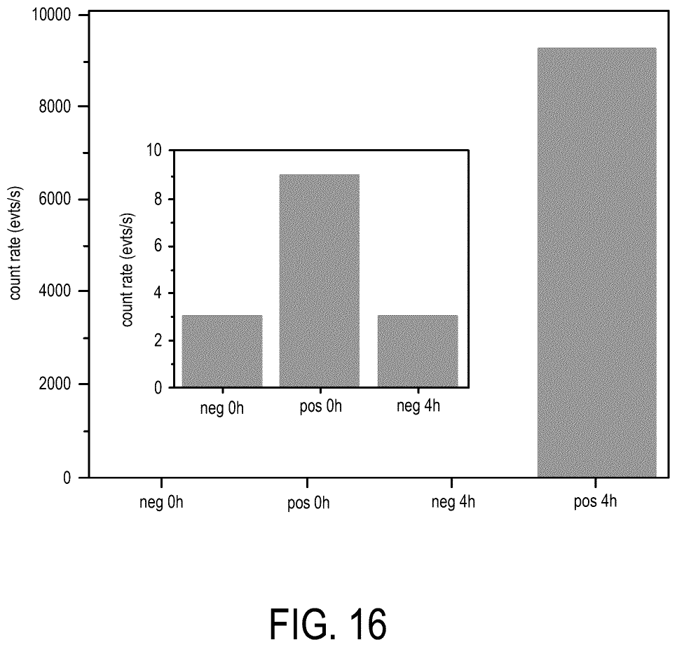

Referring to FIG. 12, the tube (1) for separating bacteria from mammalian cells comprises a proximal end (3) and a distal end (7). At the proximal end, which is considered the “bottom” when held properly for initial sample insertion, is a stopper (4), which is made of a material which can prevent the egress of separating material from the tube, but which can allow entry of a needle (5). The tube can be made from rubber, for example, which can optionally be inserted or removed from the tube (1) and through which a needle can pass. The tube can be a vacutainer tube, which can be inverted so the proximal end while allows entry of the needle is pointed downwards. An example of a vacutainer tube is shown in FIG. 2.

Referring again to FIG. 12, the tube can comprise a separating material (2). This separating material can be found at the proximal end of the tube, and can be in contact with the stopper (4). The separating material can be placed inside the tube prior to insertion of the biological sample. This separating material can form a gradient, such as a step or continuous gradient. The gradient can be made of any material which can form a gradient, such as a sugar, carbohydrate, small molecule, oligomer, or polymer dissolved or suspended in buffer, biological media or water. Specific examples of gradient media include, but are not limited to, polyhydric alcohols, sucrose, glycerol, sorbitol, dextrans, polysucrose, inorganic salts, CsCl, iodinated gradient media, Diatrizoate, Nycodenz®, Histodenz™, Iodixanol, and Percoll®. Sucrose gradient is a preferred example. FIG. 12A depicts a tube (1) with separating material (2) layered on the distal side of the stopper (4), so that the separating material is on the proximal end (3) of the tube and is in contact with the stopper (4).

As shown in FIG. 12B, a biological sample (6) can be layered on the distal side (7) of the separating material (gradient media). There are many ways that this can be achieved, and one example is to use a needle (5) and syringe (8). The biological sample (6) is drawn into the syringe (8) and then the needle (5) is plunged into the stopper (4). The needle traverses the separating material (2), and the contents of the syringe are then released on the distal side of the separating material (2) so that the biological sample is in contact with the distal end of the separating material. The separating material is sufficiently denser than the biological sample so that the biological sample lies on top of the separating material and does not penetrate into the separating material. In other words, the biological sample lies on top of the separating material and does not travel in a proximal manner into the separating material until centrifugation occurs, which is discussed below.

Biological samples for use with the methods, tubes, and kits disclosed herein include solid and semi-solid samples, such as feces, biopsy specimens, skin, nails, and hair, and liquid samples, such as urine, saliva, sputum, mucous, blood, plasma, serum, amniotic fluid, semen, vaginal secretions, tears, spinal fluid, washings, and other bodily fluids. Included among the sample are swab specimens from, e.g., the cervix, urethra, nostril, and throat. Any of such samples may be from a living, dead, or dying animal or a plant. Animals include mammals, such as humans. Other biological samples include samples of food products, animal feed, waste water, drinking water, sewage, soil, dust, and the like. For example, the biological sample can be a raw, diluted, or cultured biological fluid. Further examples of biological samples are given above in the “definitions” section.

The biological sample can also be cultured prior to introduction into the tube disclosed herein. The culture medium may be any suitable medium and may be selected according to the nature of the clinical sample and/or the suspected microorganism, and/or clinical condition of the subject, etc. Many different microbial culture media suitable for such use are known. The entire culture media can be used with the methods disclosed herein, and where this is the case, the culture media is referred to in its entirety as the “biological sample.”

The biological sample can contain, or can be suspected of containing, bacteria. The bacteria to be isolated can be infectious or non-infectious. Although any bacterial infection is encompassed, the method of the invention has particular utility in the detection or diagnosis of sepsis (or more generally management of sepsis), or where sepsis is suspected. Thus the clinical sample may be from a subject having, or suspected of having, or at risk of, sepsis. In such a case the sample will generally be blood or a blood-derived sample.

Particularly, clinically relevant genera of bacteria include Staphylococcus (including Coagulase-negative Staphylococcus), Clostridium, Escherichia, Salmonella, Pseudomonas, Propionibacterium, Bacillus, Lactobacillus, Legionella, Mycobacterium, Micrococcus, Fusobacterium, Moraxella, Proteus, Escherichia, Klebsiella, Acinetobacter, Burkholderia, Entercoccus, Enterobacter, Citrobacter, Haemophilus, Neisseria, Serratia, Streptococcus (including Alpha-hemolytic and Beta-hemolytic Streptococci), Bacteroides, Yersinia, and Stenotrophomas, and indeed any other enteric or coliform bacteria. Beta-hemolytic Streptococci would include Group A, Group B, Group C, Group D, Group E, Group F, Group G and Group H Streptococci.

Non-limiting examples of Gram-positive bacteria include Staphylococcus aureus, Staphylococcus haemolyticus, Staphylococcus epidermidis, Staphylococcus saprophyticus, Staphylococcus lugdunensis, Staphylococcus schleiferei, Staphylococcus caprae, Staphylococcus pneumoniae, Staphylococcus agalactiae Staphylococcus pyogenes, Staphylococcus salivarius, Staphylococcus sanguinis, Staphylococcus anginosus, Streptococcus pneumoniae, Streptococcus pyogenes, Streptococcus mitis, Streptococcus agalactiae, Streptococcus anginosus, Streptococcus equinus, Streptococcus bovis, Clostridium perfringens, Enterococcus faecalis, and Enterococcus faecium. Non-limiting examples of Gram-negative bacteria include Escherichia coli, Salmonella bongori, Salmonella enterica, Citrobacter koseri, Citrobacter freundii, Klebsiella pneumoniae, Klebsiella oxytoca, Pseudomonas aeruginosa, Haemophilus influenzae, Neisseria meningitidis, Enterobacter cloacae, Enterobacter aerogenes, Serratia marcescens, Stenotrophomonas maltophilia, Morganella morganii, Bacteroides fragilis, Acinetobacter baumannii and Proteus mirabilis.

Centrifugation can be used to separate the bacterial cells from the mammalian cells. During the process of centrifugation, a higher percentage of bacterial cells migrate through the separating material, while a lower percentage of mammalian cells and other material migrate through the separating material, resulting in an enriched, or concentrated, number of bacterial cells in the separating material. That separating material can then be removed and used for further processing. The details of this are discussed above.

One of skill in the art will appreciate how to carry out centrifugation in a way that results in a higher concentration of bacterial cells in the separating media. Of course, the exact time and speed of centrifugation can depend on a variety of factors, such as the concentration of bacterial cells which one desires to achieve, the amount of starting material (biological sample), the type of separating material (density gradient) used, etc. Furthermore, one of skill in the art will appreciate that similar results can be achieved either by carrying out centrifugation for a longer time at a lower speed, or at a shorter time at a higher speed. For example, the speed used can be 500, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or 10,000×g or more, less, or any amount in between these values. For example, the speed can be 1,000 to 6,000×g. In another example, the speed can be 2,500 to 4,500×g. In yet another example, the speed can be about 3,500×g. The tube can be centrifuged for about 30 seconds, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes or more, less, or any amount above, below, or in between these values. For example, the tube can be centrifuged for 30 seconds to 30 minutes, or 5-15 minutes, or about 10 minutes.

One of skill in the art will also appreciate that any type of centrifuge can be used with the methods disclosed herein, as long as it is compatible with the type of tube being used. Different adapters can be used depending on the type of tube and type of centrifuge. In one example, a swinging bucket rotor-type centrifuge can be used, and an adapter for the tube described herein can be used with it.

As mentioned above, the biological sample can be treated with a detergent which lyses mammalian cells before or after the biological sample is inserted into the tube. Because the mammalian cells are lysed while bacterial cells are not, this can result in bacterial cells migrating at a higher rate into the separating material during centrifugation. One of skill in the art will understand how to select an appropriate detergent which can preferentially lyse mammalian cells. Examples include, but are not limited to, saponin and sodium dodecylsulfate (SDS).

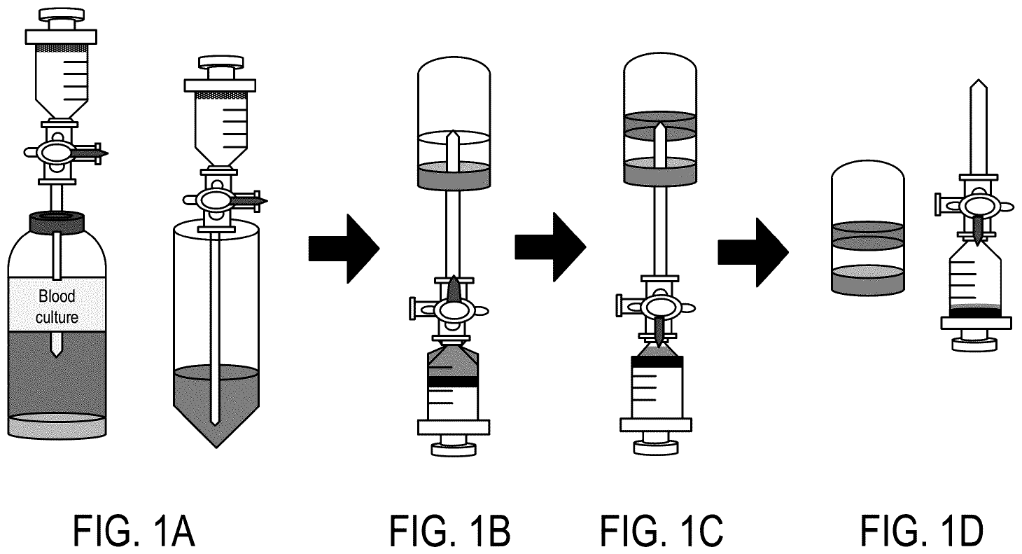

Referring to FIG. 1, a needle can be used to insert the biological sample into the tube. This needle can be attached to a syringe. The syringe and needle can be separated by a stopcock or automated pump and switch. When a stopcock is used, it can be, for example, a three-way stopcock. An example of using a three-way stopcock with the method disclosed herein is given in Example 1, but in short, with the stopcock closed to the needle, the needle can be inserted through the stopper (septum) and the separating material of the tube and deposited at the top of the separating material. The stopcock can then be opened, and the biological sample can be layered on top of the separating material. Following addition of the biological sample, the needle can be further pushed through the uppermost biological sample and the upper air evacuated (removed) through the needle. The stopcock can then be closed to the syringe and the needle completely removed from the tube.

After the centrifugation and recovery of the final sample, the sample can be used in a variety of ways. One of skill in the art will appreciate the variety of ways that the final sample can be used, such as to culture the sample, or amplify the genetic material thereof through means such as PCR, or sequence it by a variety of means, such as high-throughput sequencing. In a preferred embodiment, the final sample is subjected to an antimicrobial susceptibility test (AST). A detailed protocol for carrying out AST can be found in Bayot et al. (Bayot ML, Bragg BN. Antimicrobial Susceptibility Testing. [Updated 2021 Oct. 17]. In: StatPearls [Internet]. Treasure Island (FL): StatPearls Publishing; 2022 January, herein incorporated by reference in its entirety for its teaching concerning AST).

Further disclosed is a kit. The kit can comprise a tube for separating bacteria from mammalian cells, wherein the tube comprises a separating material, wherein a proximal end of the tube is comprised of a material which allows entry of a needle, and wherein the separating material is in the proximal end of the tube so that the separating material is in contact with the proximal end of the tube. In one embodiment, the kit comprises a syringe with a three-way stopcock, as described above. The syringe can also comprise a needle. The tube can be a vacutainer tube.

The methods disclosed herein can be implemented in a high-throughput manner. This approach can shorten the post-blood culture processing time significantly. For example, the entire process can take 5, 10, 15, 20, 25, 30, 35, 40, 45, 50, 55, or 60 minutes, or 1.5, 2.0, 2.5, 3.0, 3.5, 4.0, 4.5, 5.0, 5.5, 6.0,,5, 7, 7,5, 8, 8,5, 9, 9,5, 10, 10,5, 11, 11,5, 12, 12,5, 13, 13,5, 14, 14,5, 15, 15,5, 16, 16,5, 17, 17,5, 18, 18,5, 19, 19,5, or 20 hours or more, less, or any amount in between, above, or below these values. This approach can make use of a unique background-free bacteria recovery and quantitative flow cytometry, using, for example, 96-well plates. Analysis software can be used to make a determination of susceptibilities and MICs in a label free manner with accuracies comparable to those of commercial (slower) systems.

Therefore, disclosed herein is a high throughput recovery system and method of recovering bacteria from both infected blood and from positive blood cultures. The rapid recovery can be coupled with AST, as outlined herein, based on flow cytometry using both bacterial count rates and morphology changes upon antibiotic exposure. Bacteria can be directly recovered from whole blood and raw blood culture through selective hemolysis and inverted centrifugation through sucrose cushions with facile collection of pure bacteria without blood product contamination.

Therefore, one of the advantages of the methods disclosed herein is a faster bacterial isolation/recovery with no background. Also, a faster AST as a result of the lower background and efficient recovery is achieved. This separation/recovery enables label-free cytometric ASTs to be performed. This facile separation would also enable other techniques to be used much more quickly (e.g. microscan, Vitek-2). It is easy to implement, inexpensive, and is compatible with all existing AST methods.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. All references cited and discussed in this specification are incorporated herein by reference in their entireties and to the same extent as if each reference was individually incorporated by reference.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: Rapid, Label-Free Antibiotic Susceptibility Determined Directly from Positive Blood Culture

Bacterial bloodstream infections are a significant cause of global morbidity and mortality. Constrained by low bacterial burdens of 1-100 colony-forming-units per mL blood (CFU/mL), clinical diagnosis relies on lengthy culture amplification and isolation steps prior to identification and antibiotic susceptibility testing. The resulting>60-hour time to actionable treatment not only negatively impacts patient outcomes, but also increases the misuse and overuse of broad-spectrum antibiotics that accelerates the rise in multidrug resistant infections. Consequently, the development of novel technologies capable of rapidly recovering bacteria from blood-derived samples is crucial to human health. To address this need, disclosed herein is bacterial recovery technology from positive blood cultures that couples selective hemolysis with centrifugation through a sucrose cushion to perform rapid, background-free cytometric ASTs without long subculturing steps. Demonstrated on the most common bloodstream infection-causing bacteria: Escherichia coli, Pseudomonas aeruginosa, and Staphylococcus aureus, near-pure bacteria are rapidly recovered (≤15 minutes) with minimal user intervention. Susceptibilities of recovered bacteria are readily performed via high throughput flow cytometry with excellent agreement with much slower, standard microbroth dilution assays. Altogether, this novel direct-from-positive blood culture antibiotic susceptibility test (AST) technology enables susceptibility determinations within as little as 5 hours, post blood culture positivity.

Disclosed herein is a rapid, novel, high-efficiency bacterial recovery technology that removes essentially all blood background, while maintaining bacterial viability. This approach couples selective hemolysis with centrifugation through a sucrose cushion for rapid recovery (<15 minutes) of near-pure bacteria with minimal sample handling. As seen in Example 1, antibiotic susceptibilities of recovered bacteria were evaluated using an in-house-developed flow cytometry-based AST and benchmarked against microbroth dilution, with AST results and MIC determinations available in as little as 5 hours from blood culture positivity.

### Materials and Methods

Blood Cultures and Antibiotics. Deidentified positive BacT/alert FA PLUS aerobic culture bottles (bioMérieux, Durham, NC) with species identification and antibiogram were obtained from the Emory Investigational Clinical Microbiology Core (ICMC) and stored at 4° C. until processed. Species were selected to cover the three most common BSI-causing bacterial families that account more than half of hospital bacterial blood infections-Escherichia coli (Enterobacteriaceae), Pseudomonas aeruginosa (non-fermenting bacteria), and Staphylococcus aureus (staphylococci). Antibiotics were chosen to cover AMR threats classified by the CDC as urgent-carbapenem-resistant Enterobacteriaceae (CRE) and serious-extended-spectrum beta-lactamase (ESBL)-producing Enterobacteriaceae, multidrug-resistant (MDR) P. aeruginosa, and methicillin-resistant S. aureus (MRSA). Consequently, as part of the standard panels, gram-negative bacteria were evaluated against ceftazidime (RPI corp., Mount Prospect, IL), meropenem (Tokyo Chemical Industry, Tokyo, Japan), and tobramycin (MP Biomedicals LLC, Santa Ana, CA) for detection of ESBL, carbapenem, and aminoglycoside resistance, respectively, and S. aureus against oxacillin (Sigma-Aldrich, St. Louis, MO) for detection of MRSA.

Preparation of recovery tubes. Recovery tubes were made by injecting 2 mL of 55% sucrose (w/v) into inverted 6-mL Vacutainer plastic blood collection tubes (BD, Franklin Lakes, NJ). Sucrose solutions were prepared in volumetric glassware by dissolving D (+)-sucrose ultrapure DNAse RNAse free (VWR Life Science, Randor, PA) in cation-adjusted Mueller Hinton broth (CAMHB; BD Biosciences, San Jose, CA) with gentle heating and stirring. Following cooling, sucrose solutions were sterilized by passing the solution through a 0.2 μM Supor® PES syringe filter (Pall Corporation, Port Washington, NY) or a Nalgene Rapid-Flow 0.2 μM aPES filter (Thermo Scientific, Waltham, MA), depending on volume. 2-mL aliquots were loaded into each Vacutainer tube using a 5 mL syringe attached to a 20 G PrecisionGlide™ 1.5″ needle (BD, Franklin Lakes, NJ). Recovery tubes were stored inverted at 4° C. until utilized.

Direct recovery of bacteria from positive blood cultures (Method 1). Positive BacT/alert FA PLUS aerobic culture bottles (bioMérieux, Durham, NC) were mixed through repeated inversion, and ˜1-1.2 mL of culture media was then removed for bacterial recovery. To prevent mixing of culture media and sucrose during sample layering, a 3-way stopcock was used in the loading apparatus (FIG. 1). With the stopcock closed to the needle, the needle was inserted through the septum and the sucrose layer of the inverted recovery tube and deposited at the top of the sucrose cushion. The stopcock was then opened, and the culture media was layered on top of the sucrose. Following culture media addition, the needle was further pushed through the uppermost culture layer and the upper air evacuated through the needle. The stopcock was closed to the syringe and the needle completely removed from the recovery tube. Tubes were centrifuged inverted for 10 minutes (3,500×g) in a swinging bucket rotor (Labofuge 400, Heraeus Instruments, Hanau, Germany) and 3D-printed plastic inserts were used to secure the inverted tubes in the rotor. The bottom portion (˜1-1.3 mL) of the sucrose layer containing bacteria was recovered for analysis by inserting a fresh syringe needle through the septum of the inverted tube. To determine initial and recovered CFU, culture media and sucrose solutions were serially diluted and plated onto LB agar (Lennox; Sigma-Aldrich, St. Louis, MO). Accounting for dilution, recovery efficiencies were determined by dividing the total CFU after recovery from sucrose by the initial CFUs, each referenced to the collection volume of the positive culture media.

Saponin pretreatment for improved bacterial recovery from positive blood cultures (Method 2). Positive BacT/alert FA PLUS aerobic culture bottles (bioMérieux, Durham, NC) were mixed through inversion and 1 mL of culture media was withdrawn and mixed with 500 μL of saponin from Quillaja sp. (2.5% w/v; Sigma-Aldrich, St. Louis, MO). This 1.5-mL sample was then mixed through pulsed vortexing (10 seconds) to lyse residual intact blood cells. Resulting lysate solutions were loaded into recovery tubes as follows. With the stopcock closed to the needle, the needle was inserted through the septum of the inverted recovery tube and positioned at the top of the sucrose cushion. The stopcock was opened, 0.6-1 mL of culture media layered on top of sucrose (volume dependent on bubble formation during saponin treatment), and the upper air evacuated. The stopcock was closed to the syringe and the needle removed from the recovery tube. Tubes were centrifuged inverted for 10 minutes (3,500×g) in a swinging bucket rotor, and the bottom portion (˜0.8-1.2 mL) of the sucrose layer was removed. Culture media and recovery solutions were serially diluted and plated onto LB agar (Lennox; Sigma-Aldrich, St. Louis, MO) to determine CFUs as described above.

Influence of sucrose on bacterial growth. To determine the effects of sucrose concentration on bacterial growth, bacteria recovered from positive blood cultures following Method 2 were diluted 1-fold (i.e. undiluted), 2-fold, 5-fold, 10-fold, 20-fold, and 100-fold in fresh CAMHB within a 96-well plate. The CAMHB broth inoculated with a fresh overnight culture was used as a positive control. Following dilution, samples were incubated at 37° C. with shaking, and growth was monitored at 15-min intervals via OD₆₀₀ measurements using a plate reader (Hidex, Turku, Finland). To further investigate the effects of sucrose on bacterial growth, recovered bacteria-sucrose solutions were diluted 10-fold (optimal dilution factor) and growth was compared to control cultures inoculated with the original blood culture, an overnight culture, or colony resuspension without sucrose exposure. Growth (37° C. with shaking) was monitored via OD₆₀₀ measurements using a Hidex plate reader.

Microbroth dilution for minimum inhibitory concentrations (MIC) with and without sucrose recovery. To isolate bacteria without sucrose, culture media from positive BacT/alert FA PLUS aerobic culture bottles (bioMérieux, Durham, NC) was serially diluted and plated onto LB agar (Lennox; Sigma-Aldrich, St. Louis, MO). Bacterial suspensions were prepared by resuspending colonies in CAMHB with incubation on a MaxQ 4000 incubator shaker (Thermo Fisher Scientific, Waltham, MA) at 37° C. and ˜225 rpm for 3 hours. Bacteria recovered via Method 2 were diluted 10-fold in CAMHB. Following incubation or sucrose recovery and dilution, bacterial suspensions were adjusted to ˜0.004 OD₆₀₀ with CAMHB and a 50-μL aliquot distributed to each well of a 96-well plate containing CAMHB with and without clinically relevant antibiotics (2-fold dilution series). Gram-negative plates included: ceftazidime (3^{rd} generation cephalosporin, RPI corp., Mount Prospect, IL), cefepime (4^{th} generation cephalosporin, Chem-Impex Int'l, Wood Dale, IL), meropenem (carbapenem, Tokyo Chemical Industry, Tokyo, Japan), levofloxacin (quinolone, Alfa Aesar, Haverhill, MA), and tobramycin (aminoglycoside, MP Biomedicals LLC, Santa Ana, CA), while S. aureus plates contained: oxacillin (beta-lactam, Sigma-Aldrich, St. Louis, MO), vancomycin (glycopeptide, Santa Cruz Biotechnology, Dallas, TX), erythromycin (macrolide, Sigma-Aldrich, St. Louis, MO), clindamycin (lincomycin), and ciprofloxacin (fluoroquinolone, Sigma-Aldrich, St. Louis, MO). Antibiotic plates were incubated at 37° C. for 16-20 hours (gram-negative) or 35° C. for 24 hours (S. aureus), at which time, plates were removed from the incubator and visually inspected for bacterial growth. The MIC was set at the lowest antibiotic concentration at which no growth was detected. When visual inspection resulted in an uncertainty in bacterial density, the antibiotic concentration resulting in an OD₆₀₀ measurement<0.2 was chosen.

AST workflow using bacteria recovered directly from positive blood culture. A 1-mL aliquot of bacteria recovered using separation method 2 was diluted 10-fold in CAMHB, the OD₆₀₀ adjusted to ˜0.01, and a 50 μL aliquot distributed into each well of a 96-well plate containing CAMHB with and without ceftazidime, tobramycin, and either meropenem (E. coli and P. aeruginosa) or oxacillin (S. aureus). Following Clinical and Laboratory Standards Institute (CLSI) guidelines³³, 2% sodium chloride (NaCl) was present in wells containing oxacillin to assist in the detection of methicillin-resistant S. aureus (MRSA). Antibiotic plates were prepared such that the addition of the 50 μL bacterial suspension resulted in antibiotic concentrations corresponding to 0.25-128 μg/mL ceftazidime, 0.06-32 μg/mL meropenem, 0.125-64 μg/mL tobramycin, and 0.03-16 μg/mL oxacillin (2-fold dilution series). To account for observed differences in doubling and lag times, E. coli and P. aeruginosa were incubated for 4 hours at 37° C., while S. aureus was incubated for 9 hours at 35° C. After incubation, antibiotic plates were removed from the incubator and directly stored on ice (i.e. no post-processing) until high-throughput flow cytometry (BD LSRFortessa with HTS-system, BD, Franklin Lakes, NJ). No-antibiotic controls and antibiotic-treated samples were handled identically.

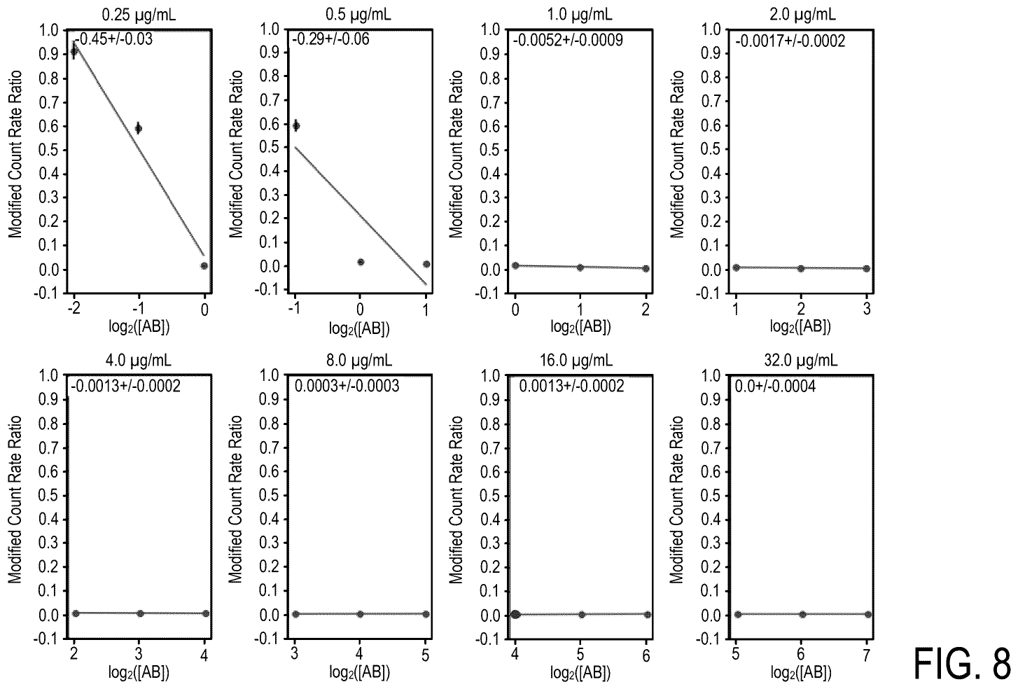

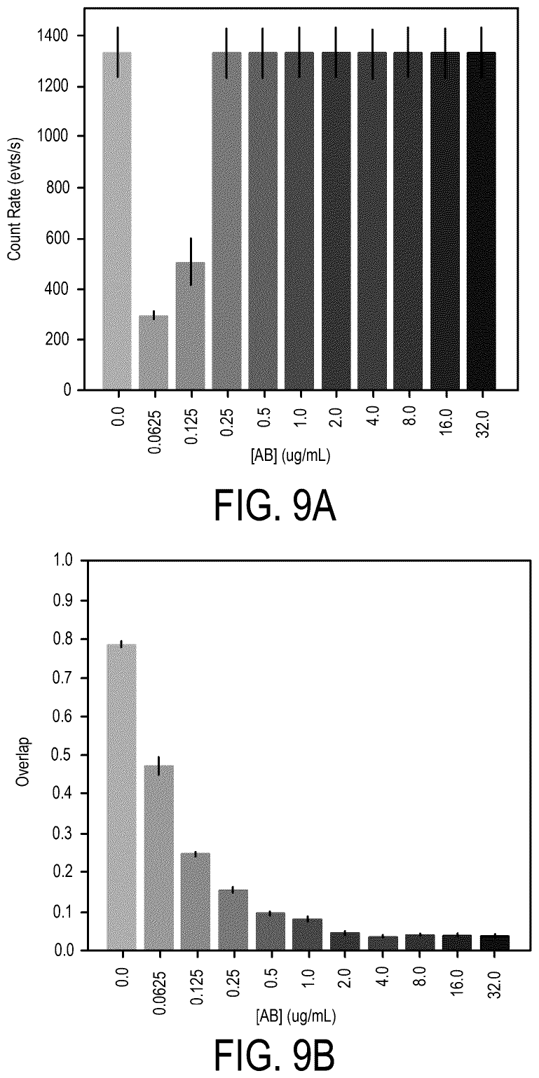

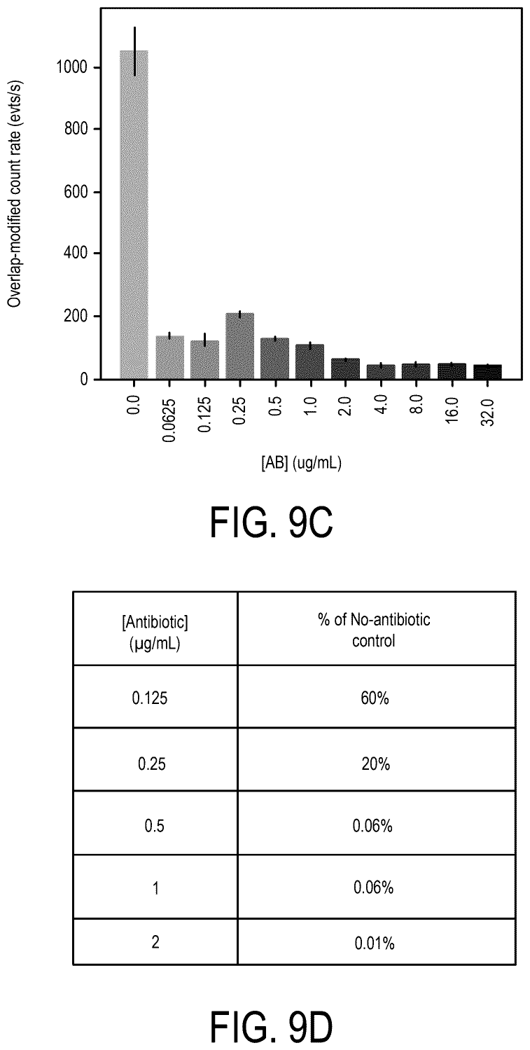

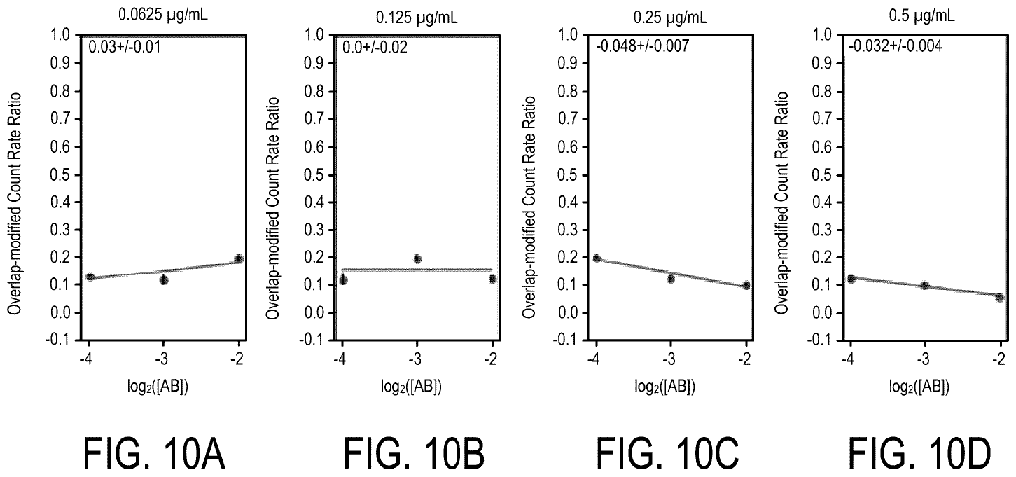

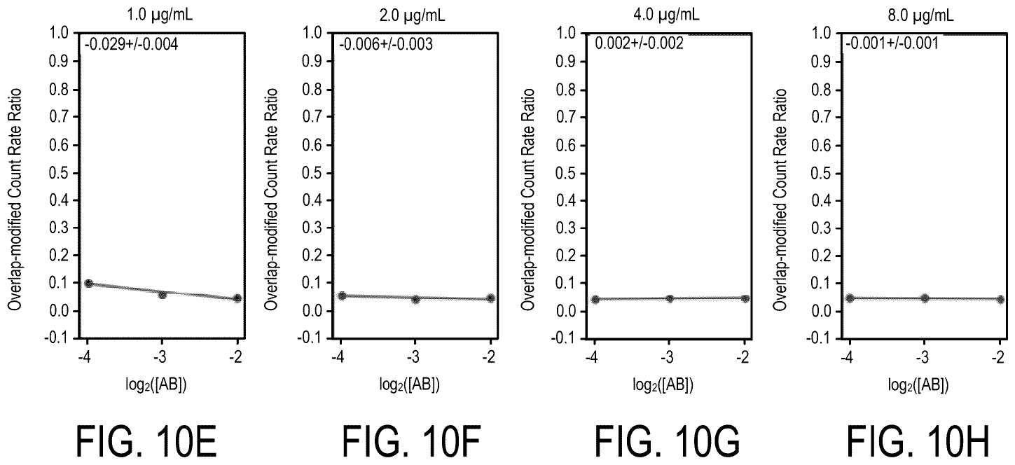

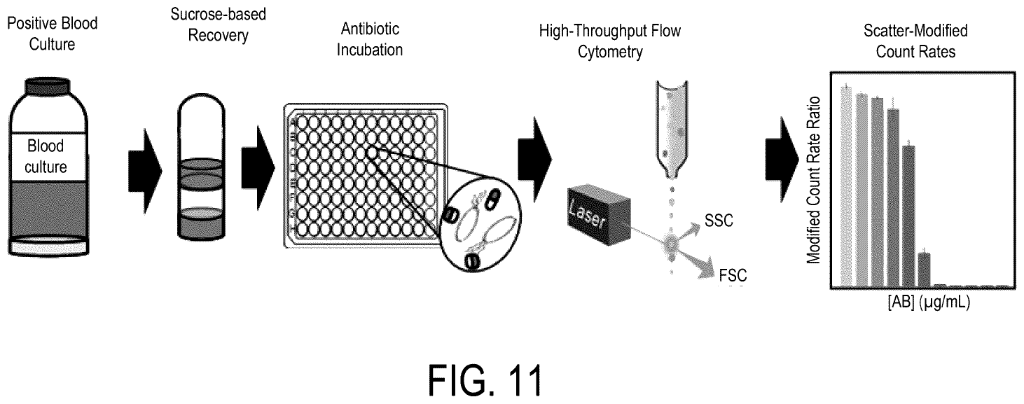

MIC Determination by flow cytometry. Because antibiotic effects are observed in both the position of scatter signatures (morphology changes) and count rates (growth inhibition), a modified count rate, accounting for scatter position, was used to evaluate susceptibility. Briefly, no-antibiotic control populations (triplicate) were pooled, binned, and a convex hull boundary set at 1-standard deviation above the background (not including bins with zero counts) was drawn. Scatter events outside this boundary of the paired no-antibiotic control (i.e. scatter events that resulted in at least one negative cross product with the boundary) were excluded. The overlap ratio, measuring changes in morphology, was then computed by dividing the number of sample events within the no-antibiotic control boundary by the total number of events within the sample. As an increase in count rate attributed to viable bacteria in antibiotic-treated samples is not scientifically meaningful, the fractional overlap was multiplied by the minimum of the control and sample count rates to generate a modified count rate accounting for antibiotic-induced morphological changes. All modified count rates were normalized by the average of the no-antibiotic controls. For automatic MIC determination, normalized count rates corresponding to n (index of predicted MIC), n+1, n+2 were iteratively fit to a linear function with a frameshift of n+1 between iterations (FIGS. 8 and 9). 3-point fits of antibiotic samples with normalized count rates less than 0.5 that resulted in a slope greater than or equal to −0.01 (within error) were classified as sensitive. A threshold of −0.01 instead of zero was used to mitigate the influence of small fluctuations in normalized count rates on susceptibility determinations. The MIC was set as the lowest antibiotic concentration that resulted in a consistent slope of ≥−0.01 (within error) for all subsequent 3-point linear fits (i.e., higher antibiotic samples).

In-line Gram staining with flow cytometry. To rapidly identify whether a Gram positive or Gram negative antibiotic panel should be used, and potentially better classify any unknown pathogen isolated from positive blood culture, an in-line Gram stain compatible with flow cytometry was employed. The Live Baclight Bacterial Gram Stain Kit (Thermo Fisher Scientific, Waltham, MA) makes use of a two-color dye cocktail to differentially stain Gram positive and negative bacteria using a 1:1 mixture (by volume) of 3.34 mM SYTO9 and 4.67 mM hexidium iodide (HI) solutions. The slight concentration discrepancy of the pure dye addresses the average differential uptake of dye by Gram-positive vs Gram-negative bacteria. Prior to fluorescent dye labeling, recovered bacteria were adjusted to ˜0.01 OD₆₀₀, spun down for 3 minutes at 2048×g at 4° C. (Centrifuge 5417R, Eppendorf) and resuspended in PBS. 97 μL of this suspension was added to individual wells of a 96-well plate. Plates can be stored at 4° C. until use. 15 minutes prior to measuring on the flow cytometer, 3 μL of the Baclight dye cocktail was added and allowed to incubate at room temperature in the dark to allow for dye uptake. In addition to the normal forward and side scatter channels, the SYTO9 and hexidium iodide emission were measured on the FITC 530/30 nm and Texas Red 610/20 nm fluorescence channels, respectively (BD LSRFortessa with HTS-system, BD, Franklin Lakes, NJ).

Support Vector Machine (SVM) training. Known Gram positive and Gram negative bacterial strains were stained with the Baclight dye cocktail described above to train the support vector machine (SVM) classifier implemented in sci-kit learn in python 3.8.5. Once trained, the SVM classifier identifies the optimal boundary separating Gram positive and Gram negative strains based on the two-dimensional space constructed from the two fluorescence channels. The optimal SVM boundary is identified via grid search that iterates over initial parameters to optimize sensitivity and precision scores. Cross validation is performed by splitting the training data into k-subsamples, allowing for k−1 subsamples to be used for training the SVM with grid search. The remaining subsample is then be used to score the model and provide the means to compare all of the models over which grid search is optimized. This k-fold cross validation reduces model overfitting by providing a “fresh”′ set of training and testing data with each k-fold cut.

### Results and Discussion

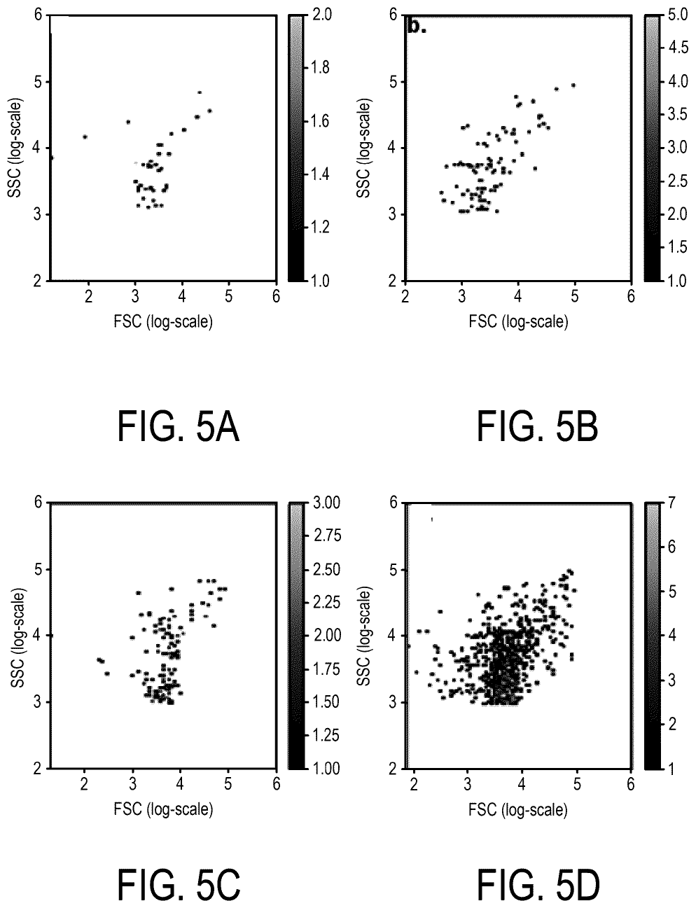

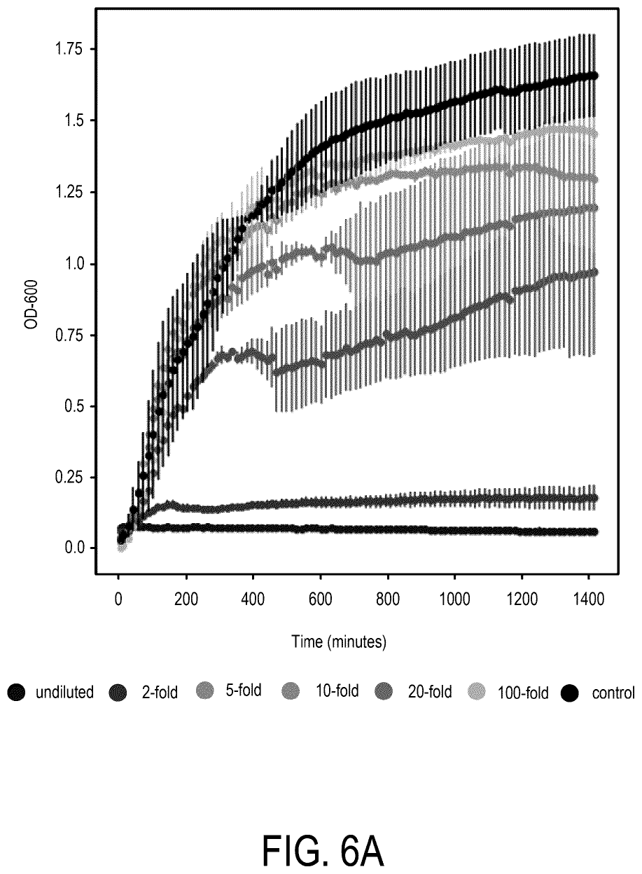

Incorporation of inverted sucrose tubes for improved recovery. Due to osmotic pressure and the higher permeability of bacterial relative to mammalian membranes, bacterial cell densities increase more in sucrose than do those of (lysed or intact) blood cells. Consequently, the need for clean recovery of bacteria necessitated the use of an inverted tube configuration for collecting bacteria. Dorn, et. al,²² utilized a different approach for blood culture by centrifuging infected lysed blood in inverted Vacutainer tubes containing a sucrose-gel to recover bacteria. While the inverted tube enables direct recovery of bacteria (i.e. collect through the septum from the “bottom” of the inverted tube), the gel not only adds increased processing time and protocol complexity, but it is difficult to handle and injection of blood through this sucrose-gel mixture prior to centrifugation left a needle tract in the sucrose-gel that prevented clean separation of bacteria from blood. Further, the gel significantly increased scatter background (FIG. 5), preventing flow cytometric analyses. Consequently, the gel was eliminated, and instead, a pure sucrose cushion was used for separations, with sample loading being achieved using a 3-way stopcock to prevent layer mixing upon loading (FIG. 1). These separations therefore improve on the approach of Dorn et al. as recovered bacteria through pure sucrose is fully compatible with light scattering based-ASTs (i.e. turbidity and flow cytometric) and employs a simplified recovery protocol with limited user intervention and no need for plating before bacterial recovery.

Using pure sucrose cushions for recovery, culture media is collected directly from the positive blood culture tubes (FIG. 1, al) or following 10-second saponin pretreatment (FIG. 1, a2) with the stopcock closed to the perpendicular port. Prior to needle insertion into the septum (bottom) of the BD Vacutainer tube, the stopcock is closed to the needle to prevent mixing of the culture media and sucrose layers. Once the needle is positioned at the top of the sucrose cushion, the stopcock is opened and the culture media is layered on top (FIG. 1b). To prevent the lid from popping off during centrifugation, the upper air is evacuated through the needle and the stopcock closed to the syringe prior to removing the needle (FIG. 1c). Following sample loading, the tube is kept inverted through centrifugation and recovery. As depicted in FIG. 2 a&c, clean layering of positive blood culture onto sucrose cushions is achieved following this separation protocol.

Recovery Performance of inverted tubes containing sucrose. To evaluate the recovery performance of inverted BD Vacutainer tubes containing 55% sucrose (w/v), positive blood cultures containing E. coli, P. aeruginosa, and S. aureus were processed as schematized in FIG. 1. Recovery with and without saponin pretreatment was also evaluated as a means to improve recovery. Following sample loading, tubes were centrifuged inverted for 10 minutes (3,500×g) and the bottom portion of the sucrose cushion recovered. Bacteria, before and after separation were serially diluted and plated to determine CFU/mL, accounting for dilution factor and sample volume. Recovery efficiencies were calculated by dividing the CFU/mL recovered in sucrose by the initial CFU/mL in the blood cultures prior to sucrose separation. As reported in Table 1, bacterial recovery without saponin pretreatment is quite low (˜0.1-1%) for all bacteria studied, likely due to the formation of a red blood cell (RBC) plug inhibiting bacterial migration into the sucrose layer (FIG. 2b, dark red layer at interface). While decreased recoveries are observed, Enterobacteriaceae demonstrate sufficient bacterial CFU and growth (following a modest enrichment period) for strong signal in downstream antibiotic susceptibility determinations. However, the decreased P. aeruginosa signals observed during AST analysis necessitates improved recovery yield to limit downstream incubation periods. Incorporation of saponin pretreatment (10 second vortex) enables a ˜10-fold improvement on recovery of S. aureus and ˜100-fold improvement on E. coli and P. aeruginosa as shown in Table 1, as residual RBCs are lysed and the RBC plug no longer forms (FIG. 2d). Thus, using saponin consistently enables>10% recovery efficiencies directly from positive blood cultures.

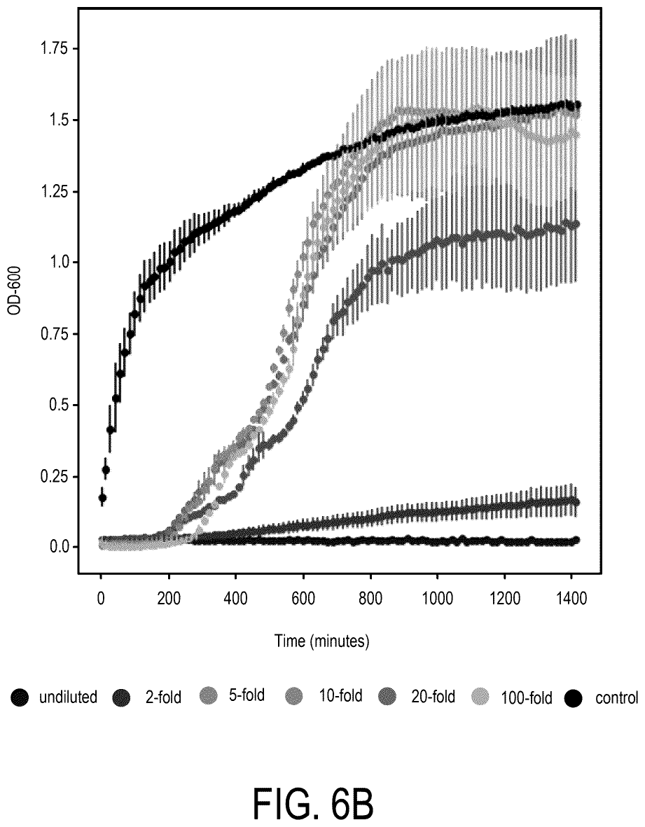

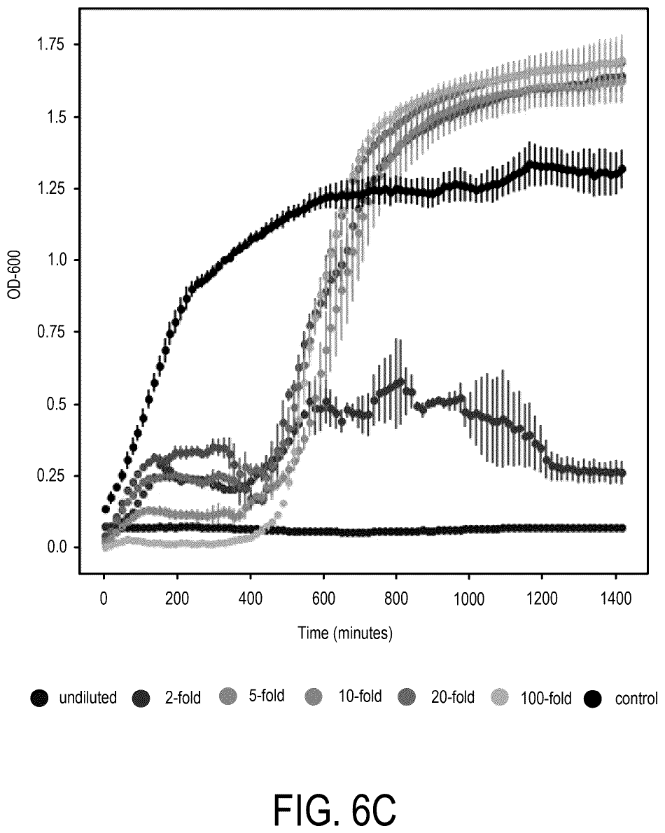

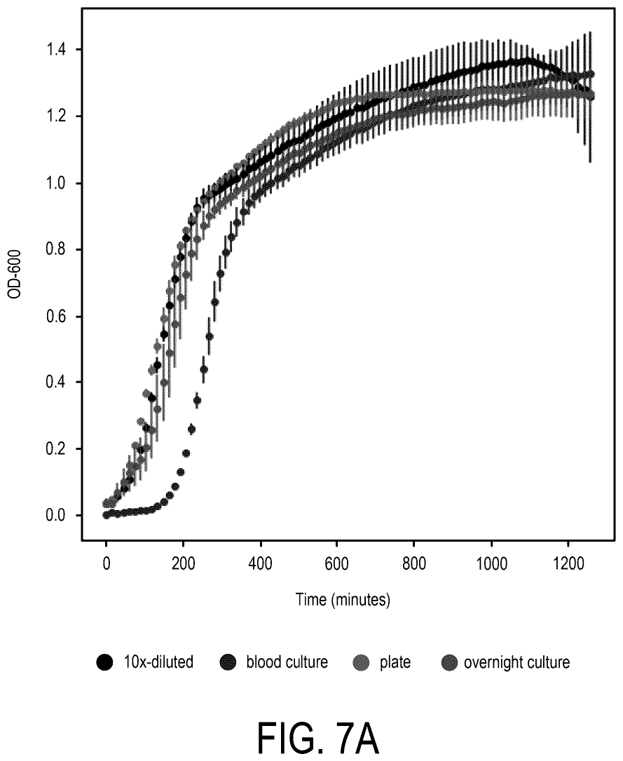

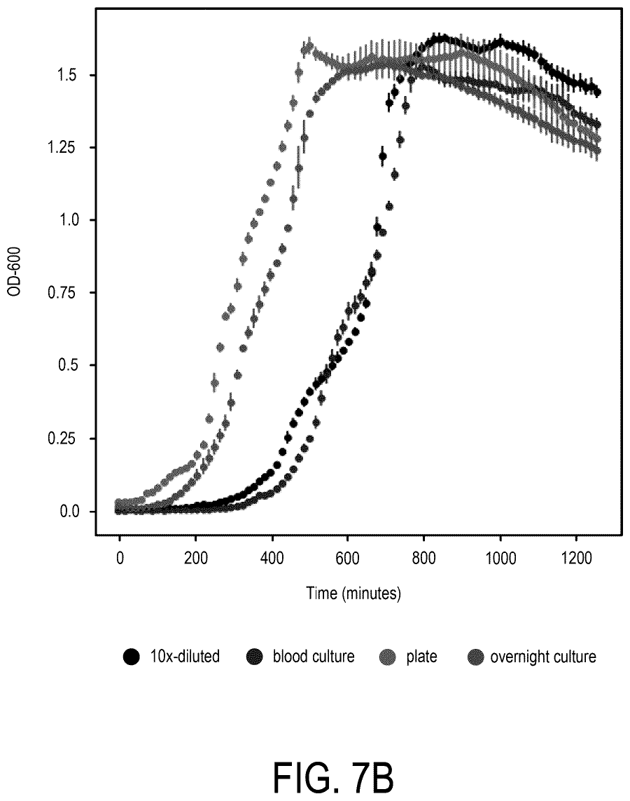

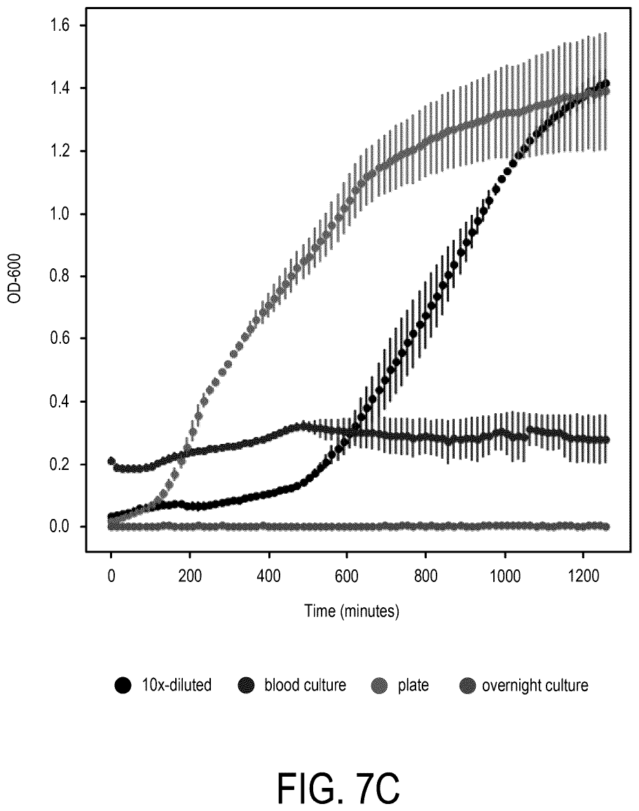

Impact of sucrose on bacterial growth. As high sucrose concentrations can lead to bacterial growth inhibition through limiting water exposure and osmotic shrinkage/stress,³⁴ the influence of sucrose concentration on bacterial growth following recovery was studied. Recovered bacteria within 55% sucrose (w/v) were directly or 2-fold (27.5%), 5-fold (11%), 10-fold (5.5%), 20-fold (2.75%), and 100-fold (0.55%) diluted in CAMHB and growth monitored on a plate reader set at 37° C. with medium shaking. Growth of the pure isolate (no sucrose) was used as a control. Assessment of the growth curves of E. coli (FIG. 6a), P. aeruginosa (FIG. 6b), and S. aureus (FIG. 6c) in various sucrose concentrations and with no previous sucrose exposure (black curve) suggest that bacterial growth becomes increasingly hindered as sucrose concentration increases, with 55% sucrose inhibiting growth completely (i.e., flat curve). Consequently, dilution of sucrose prior to the phenotypic AST is necessary to promote recovery of bacterial growth. To better understand the effects of sucrose exposure on bacterial growth, growth of 10× diluted recovery solutions (optimal dilution factor) were compared to bacteria at different stages along standard AST workflows. (i.e. present within the original blood culture, plate resuspension, and overnight broth culture). Comparison of growth curves (FIG. 7) suggests the increased lag-time observed in recovery solutions is caused by the initial state of bacteria within the positive blood culture and not the sucrose exposure, with recovery solutions of E. coli (FIG. 7a) even demonstrating decreased lag times in 10-fold diluted sucrose relative to those in diluted positive blood culture.

Influence of Sucrose on Bacterial MIC. To rule out the potential effect of sucrose exposure in bacterial antibiotic susceptibility, microbroth dilution studies were performed on bacteria recovered using standard practices (i.e. plating following positive blood culture) and through selective lysis coupled with centrifugation through the sucrose cushion (Method 2). As shown in Tables 4-6, 100% agreement in susceptibility classification is observed for bacteria recovered through standard plating and Method 2, indicating that the limited exposure to increased sucrose concentrations has no effect on downstream susceptibility determinations.

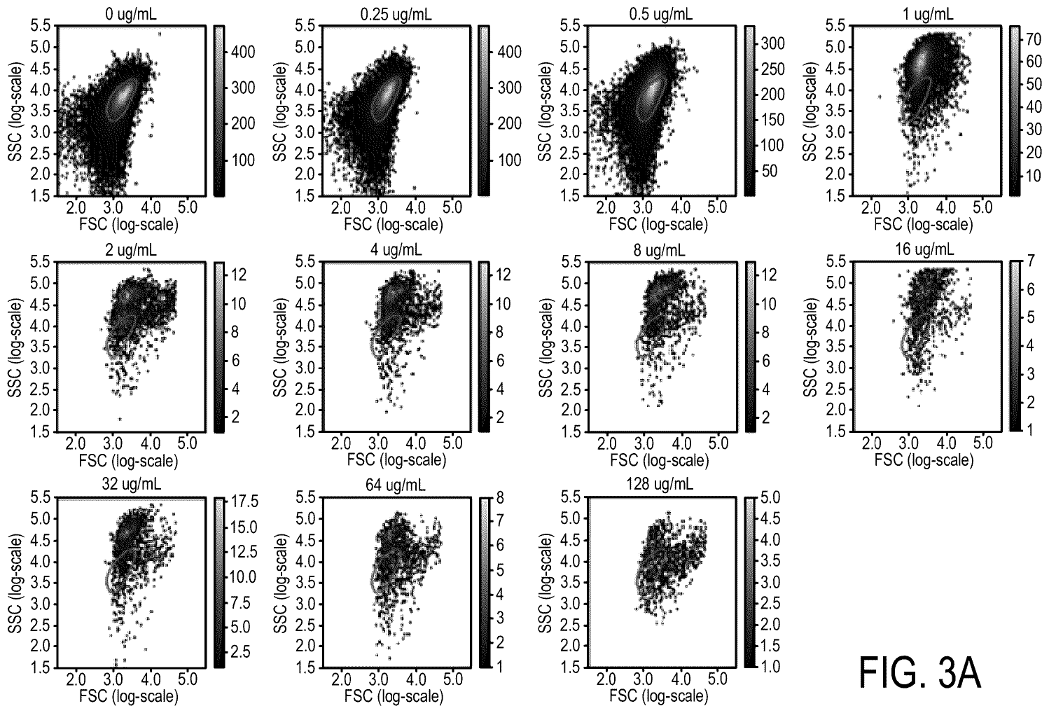

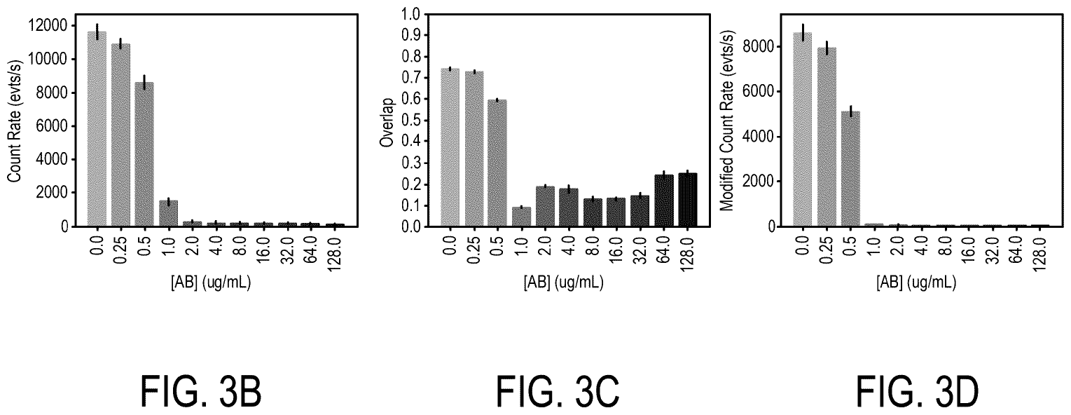

Direct, flow cytometric-based AST for antibiotic susceptibilities. Despite its ability to rapidly examine individual cell morphology and physiology within a large heterogeneous population, flow cytometric-based ASTs have largely relied on fluorescence viability tests.^{25, 35-39} Not only do such viability tests demonstrate signal overlap and background issues, but they also suffer from increased failure rates when evaluating certain bacteria-antibiotic combinations,³⁷⁻³⁹ thereby limiting their application in clinical settings. Consequently, label-free approaches are clinically attractive. Without the added information provided by fluorescence, analysis of near-pure bacteria is crucial to prevent obstruction of bacterial signals by blood background. As our separation technology removes essentially all blood background, susceptibility determinations of bacteria recovered from positive blood cultures and those recovered from overnight cultures of pure isolates were compared in our flow cytometric-AST. Positive blood cultures (overnight cultures) containing E. coli, P. aeruginosa, and S. aureus were separated using selective hemolysis coupled with sucrose-centrifugation. Recovered solutions (containing near-pure bacteria) were 10-fold diluted in CAMHB to enable restoration of bacterial growth. Diluted bacterial sucrose solutions were adjusted to an optical density of ˜0.01 OD₆₀₀ and 50 μL was dispensed into each well within the AST panel to evaluate susceptibilities. Of all the samples evaluated, only one E. coli strain did not produce an optical density≥0.01 following the 10-fold dilution. Antibiotic plates were incubated for 4 hours at 37° C. (gram-negative) or 9 hours at 35° C. (S. aureus), removed from the incubator, and stored on ice until high-throughput flow cytometry was performed. Of the 22 strains tested, only one P. aeruginosa strain did not demonstrate significant growth within the antibiotic incubation period (no-antibiotic control count rate<100 events/s); while growth was detected in positive control wells of broth microdilution. Evaluation of flow cytometric signals of untreated and antibiotic-treated bacteria (FIG. 3) shows that changes in both count rate (FIG. 3B) and scatter position (FIGs. 3A&C) occur as the MIC is approached. Because changes in both count rate (growth inhibition) and scatter signatures (morphology) report on susceptibility, sample count rates were adjusted for scatter position by incorporating overlap of the scattered light distributions (FIG. 3D) and normalized by the no antibiotic control counts prior to assessment of antibiotic susceptibility. All sensitive bacteria show reduced CFU/mL above their MICs, but morphology changes and increased scatter signals can occur as the MIC is approached. Combining morphology and count rate changes relative to the no-antibiotic control (see Methods), enables highly accurate susceptibility and MIC determinations for all bacteria/antibiotics studied. Automated susceptibility determinations were performed via iterative three-point linear fitting of morphology-modified count rate as a function of log 2 (antibiotic concentration) with the MIC set as the lowest antibiotic concentration which maintained a slope≥−0.01 for all subsequent 3-point fittings (FIG. 8 1 μg/mL and S6 0.25 μg/mL). When the scatter-modified count rate ratio (y-axis of FIG. 3C) of all antibiotic concentrations was ≤0.1 or ≥0.5, bacteria-antibiotic pairs were automatically categorized as sensitive and resistant, respectively. Interestingly, 4-hour treatment of P. aeruginosa with ceftazidime and meropenem can demonstrate an initial decreased count rate as the MIC is approached then an increase at or near the MIC. Evaluation of the scatter positions shows limited overlap at this point to suggest the increase in count rate is not attributable to viable bacteria (confirmed by plating). Increasing the antibiotic incubation time to 7 hours mitigates this effect, yielding trends consistent with faster growing E. coli. Nonetheless, selecting the MIC as the lowest antibiotic concentration that results in a continued slope of ≥−0.01 for all subsequent 3-point fittings (i.e., higher antibiotic concentrations) following 4-hour incubation with these antibiotics enables faster reporting times to yield strong agreement with BMD.

Susceptibility classifications of sensitive, intermediate, and resistant (Tables 2-5) for bacteria that demonstrated growth within the antibiotic incubation period show a ˜90% categorical agreement between methods, within the standard 2-fold error tolerance. Of the five misclassifications, one resulted from a sensitive vs. intermediate result between our novel AST and broth microdilution, one an intermediate versus resistant result, one a resistant versus sensitive, and two from a sensitive versus resistant result. P. aeruginosa was responsible for both of the sensitive versus resistant results. MIC determinations show ˜73% accuracy with broth microdilution MICs.

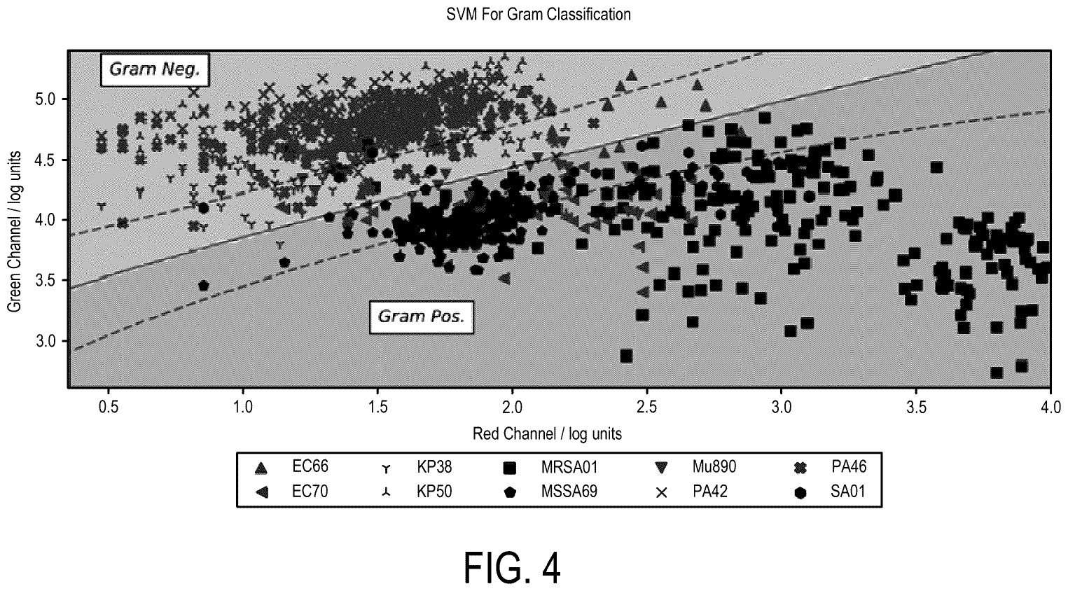

Rapid in-line cytometric Gram staining. Because of the growth and antibiotic panel differences in assessing Gram positive vs. Gram negative bacterial susceptibilities, a fast, inline Gram stain adds important information to clinical analysis. The clean bacterial recovery achieved with sucrose-based separations enables a rapid cytometry-based Gram stain to be readily performed prior to the cytometric AST. Upon differential labelling Gram positive and Gram negative bacteria with Baclight dyes (methods), a simple SVM-based classifier directly and rapidly discerns the Gram status of each recovered bacterial cell with 94% precision and sensitivity. Comparison of the two fluorescence channels of these dye-laden bacteria yields a two-dimensional space that maximizes the discrimination between Gram positive and negative bacterial classes (FIG. 4). The solid black line in FIG. 4 is the optimal boundary separating Gram positive and negative bacteria as determined by the grid-search and k-fold cross-validation algorithm (methods). This trained model can be used to rapidly assess the Gram status of newly isolated pathogens to inform growth and AST panel conditions.

High Throughput Means. High throughput flow cytometry in multi-well plates is uniquely enabled by the clean bacterial recovery and near complete background rejection. ASTs are readily completed within 5 hours of blood culture positivity with excellent accuracy for both gram negative and gram positive bacteria. As pure bacteria are recovered through this process, rapid identification can also be performed in parallel with ASTs, without additional subculturing being needed. This approach requires minimal sample handling and lab technician labor, resulting in an approach that is wholly compatible with clinical microbiology lab work flows. The unique and complete background rejection yields full AST results more than 1 day faster than current clinical methods allow.

The exemplary rapid recovery system and method can be used for recovery of essentially pure bacteria from blood, blood culture, and other bodily fluid/patient samples, and their cultures. The purification facilitates direct analysis of recovered bacteria with flow cytometry for cytometric-based antimicrobial susceptibility testing and potential identification. The rapid and background-free isolation from blood and other dirty patient samples through sucrose cushions in a unique process and geometry facilitates fast susceptibility tests. Thus, >10% of bacteria can be readily recovered from patient samples, without any mammalian cell background. The exemplary system and method facilitate direct ASTs to be performed very soon after blood cultures indicate the presence of bacteria. Bacteria recovery can be done before cultures register as positive, and background rejection of this approach enables faster ASTs to be performed. Prior ASTs require subculturing that can take a total time of ˜24-36 hours after positive blood culture. The exemplary system and method can reduce the processing time to about 5 hours from a positive blood culture, precisely because of the background rejection of our separation. This technology is significantly different than AST, which involves Fastinov (which is also flow cytometry based, but uses fluorescent dyes for measuring uptake and cannot determine minimum inhibitory concentrations). Cytometric AST is label-free, faster due to the novel purification/separation, and can determine MICs with good accuracy. This approach is also much faster than non-cytometry-based methods currently used in clinical microbiology labs (Vitek-2 and microscan). Again, the separation precludes the need for subculturing of bacteria and uniquely enables our novel cytometry-based AST without dyes to measure susceptibility profiles and MICs.

The methods disclosed herein allow for the appropriate antibiotic treatment for any bacterial infection in a variety of patient samples.

### CONCLUSIONS

By coupling selective hemolysis with centrifugation through inverted sucrose tubes, recovery of near-pure bacteria from positive blood culture media is achieved in ≤15 minutes with minimal sample processing. Broth microdilution analysis of bacteria recovered using this novel approach, as well as, standard plating yield MICs within the normal 2-fold error range suggesting its compatibility with clinical workflow for results 1 day faster than currently possible. Furthermore, evaluation of antibiotic-induced changes in flow cytometric count rate, accounting for scatter position, enables 90% true susceptibility determinations within as little as 5 hours, post blood culture positivity. Improvement to susceptibility and MIC determinations may be achieved by real-time monitoring of bacterial growth to ensure strong bacterial signals during sample acquisition and thus sufficient antibiotic exposure. Overall, this direct flow-cytometric AST portends significant improvement to patient outcomes and antimicrobial stewardship by shortening the time to administration of actionable treatments to as little as 5 hours and 10 hours post blood culture positivity for Gram-negative and S. aureus infections, respectively. The addition of an inline cytometric Gram stain and SVM-based classifier is also uniquely enabled by the essentially complete blood background rejection and >10% bacterial recovery to rapidly define the expected incubation time and antibiotic panel to be used.

### TABLES

TABLE 1
Influence of saponin pretreatment on recovery of bacteria
from positive blood culture tubes with sucrose

| | Gram | Saponin | Recovery |
|---|---|---|---|
| Species | Status | Pretreatment | (%) |
| Escherichia coli | Neg. | No | 0.1 ± 0.1 |
| | | Yes | 14 ± 8 |
| Pseudomonas aeruginosa | Neg. | No | 0.05 ± 0.01 |
| | | Yes | 13 ± 4 |
| Staphylococcus aureus | Pos | No | 1 ± 0.3 |
| | | Yes | 13 ± 2 |

Positive blood culture with (+) and without (−) saponin pretreatment were loaded onto sucrose cushions and centrifuged for 10 minutes (3,500 xg). The bottom portion of the sucrose layer (devoid of bulk blood background) was recovered, diluted, and plated to determine cfu. Average and standard deviation are reported for 3 recovery tubes of the same blood culture bottle, with percent recovery calculated by dividing the cfu recovered in sucrose by the starting cfu collected from the blood culture.

TABLE 2
Minimum inhibitory concentrations and susceptibility classification of E. coli

| | Ceftazidime | | | | Meropenem | | | | Tobramycin | | | |
|---|---|---|---|---|---|---|---|---|---|---|---|---|
| | (μg/mL) | | | | (μg/mL) | | | | (μg/mL) | | | |
| | HTFC | | BMD | | HTFC | | BMD | | HTFC | | BMD | |
| EC37 | 8 | (I) | 32 | (R) | ≤0.06 | (S) | ≤0.06 | (S) | 0.25 | (S) | 0.25 | (S) |
| EC-bank | 16 | (R) | 32 | (R) | ≤0.06 | (S) | 2 | (I) | ≤0.125 | (S) | 0.5 | (S) |
| Mu76 | 1 | (S) | 2 | (S) | 4 | (R) | 0.5 | (S) | 0.5 | (S) | 0.5 | (S) |
| Mu218 | 16 | (R) | 32 | (R) | ≤0.06 | (S) | ≤0.06 | (S) | 16 | (R) | 32 | (R) |
| Mu890 | ≤0.25 | (S) | 0.5 | (S) | ≤0.06 | (S) | ≤0.06 | (S) | 0.5 | (S) | 0.5 | (S) |
| Blood Culture A | 8 | (I) | 16 | (R) | ≤0.06 | (S) | ≤0.06 | (S) | 0.25 | (S) | 1 | (S) |
| Blood Culture B | 32 | (R) | 64 | (R) | ≤0.06 | (S) | ≤0.06 | (S) | 4 | (S) | 8 | (I) |

Sucrose-recovered E. coli was treated with antibiotic for 4 hours (37° C.) and analyzed with high-throughput flow cytometry.
HTFC: high-throughput flow cytometry; BMD: broth microdilution; (S) sensitive; (I) intermediate; (R) resistant.
~86% accuracy (18/21) in categorical agreement and ~75% accuracy in MIC prediction (15/20) between methods, within the standard 2-fold error tolerance.

TABLE 3
Minimum inhibitory concentrations and susceptibility classification of P. aeruginosa

| | Ceftazidime | | | | Meropenem | | | | Tobramycin | | | |
|---|---|---|---|---|---|---|---|---|---|---|---|---|
| | (μg/mL) | | | | (μg/mL) | | | | (μg/mL) | | | |
| | HTFC | | BMD | | HTFC | | BMD | | HTFC | | BMD | |
| PA7 | 8 | (S) | 128 | (R) | 2 | (S) | 1 | (S) | >16 | (R) | >64 | (R) |
| PA46 | 2 | (S) | 1 | (S) | 1 | (S) | 0.25 | (S) | 1 | (S) | 0.5 | (S) |
| PA49 | 4 | (S) | 2 | (S) | 0.5 | (S) | 0.5 | (S) | 1 | (S) | 0.5 | (S) |
| PA81 | 8 | (S) | 4 | (S) | 8 | (R) | 8 | (R) | 1 | (S) | 0.5 | (S) |
| PAO1 | 1 | (S) | 1 | (S) | 2 | (S) | 0.5 | (S) | 2 | (S) | 0.5 | (S) |
| Blood Culture A | insufficient growth in 4-hour test period | | | | | | | | | | | |
| Blood Culture B | 4 | (S) | 4 | (S) | 2 | (S) | 8 | (R) | 0.25 | (S) | 2 | (S) |

| | | | | | | | | | | | |
|---|---|---|---|---|---|---|---|---|---|---|---|
| Blood Culture C | 2 | (S) | 1 | (S) | 0.125 | (S) | 0.25 | (S) | S | ≤0.125 | (S) |

Sucrose-recovered P. aeruginosa was treated with antibiotic for 4 hours (37° C.) and analyzed with high-throughput flow cytometry.
HTFC: high-throughput flow cytometry; BMD: broth microdilution; (S) sensitive; (I) intermediate; (R) resistant.
Of bacteria that demonstrated sufficient growth within the 4-hour incubation period, there is an ~90% accuracy (19/21) in categorical agreement and ~71% accuracy in MIC prediction (15/21) between methods, within the standard 2-fold error tolerance.

TABLE 4
Minimum inhibitory concentrations and susceptibility
classification of S. aureus

| Oxacillin | | |
|---|---|---|
| (μg/mL) | | |
| | HTFC | BMD |

| | | | | |
|---|---|---|---|---|
| ATCC25923 | 0.25 | (MSSA) | 0.125 | (MSSA) |
| ATCC29213 | 2 | (MSSA) | 0.25 | (MSSA) |
| ATCC43300 | >4 | (MRSA) | 16 | (MRSA) |
| NRS382 | >4 | MRSA | >16 | (MRSA) |
| NRS385 | >4 | MRSA | >16 | (MRSA) |
| Blood Culture A | 0.125 | (MSSA) | 0.125 | (MSSA) |
| Blood Culture B | 4 | (MRSA) | >16 | (MRSA) |

Sucrose-recovered S. aureus was treated with oxacillin for 9 hours (35° C.) and analyzed with high-throughput flow cytometry. HTFC: high-throughput flow cytometry; BMD: broth microdilution; (MSSA) methicillin-sensitive S. aureus; (MRSA) methicillin-resistant S. aureus. 100% accuracy (7/7) in categorical agreement and ~71% accuracy in MIC prediction (5/7) between methods, within the standard 2-fold error tolerance.

TABLE 5
Recovery of simulated positive blood cultures
containing reduced CFU bacteria

| | Concentration | Recovery |
|---|---|---|
| Species | (CFU/mL) | (%) |
| Escherichia coli | 2.8 × 10⁶ ± 4.2 × 10⁵ | 16 ± 3 |
| | 3.4 × 10⁴ ± 8.3 × 10³ | 6 ± 6 |
| | 420 ± 53 | 12 ± 8 |
| Pseudomonas aeruginosa | 9.4 × 10⁶ ± 9.1 × 10⁵ | 11 ± 3 |
| | 1.2 × 10⁵ ± 1.4 × 10⁴ | 9 ± 4 |
| | 1500 ± 130 | 8 ± 2 |
| Staphylococcus aureus | 1.1 × 10⁵ ± 5.5 × 10³ | 9 ± 2 |
| | 1200 ± 120 | 10 ± 2 |

TABLE 6
Effects of sucrose-based recovery on minimum inhibitory concentrations
(MICs) of E. coli 79 against a variety of antibiotic classes.

| | AB Class | Standard | Sucrose |
|---|---|---|---|
| Ceftazidime | 3^{rd} gen. cephalosporin | I | I |
| Meropenem | carbapenem | ≤ 1/32S | ≤ 1/32S |
| Tobramycin | aminoglycoside | ¼S | ¼S |
| Cefepime | 4^{th} gen. cephalosporin | 2R | R |
| Levofloxacin | quinolone | >4R | 4R |

TABLE 7
Effects of sucrose-based recovery on minimum inhibitory
concentrations (MICs) of P. aeruginosa 77
against a variety of antibiotic classes.

| | AB Class | Standard | Sucrose |
|---|---|---|---|
| Ceftazidime | 3^{rd} gen. cephalosporin | ½S | ¼S |
| Meropenem | carbapenem | ⅛S | 1/16S |
| Tobramycin | aminoglycoside | ⅛S | ⅛S |
| Cefepime | 4^{th} gen. cephalosporin | ½S | ¼S |
| Levofloxacin | quinolone | ¼S | ¼S |

TABLE 8
Effects of sucrose-based recovery on minimum inhibitory concentrations
(MICs) of S. aureus 78 against a variety of antibiotic classes.

| | AB Class | Standard | Sucrose |
|---|---|---|---|
| Oxacillin | Penicillinase-stable | 4R | 4R |
| | penicillins | | |
| Clindamycin | lincosamides | 4R | 4R |
| Ciprofloxacin | fluoroquinolones | ≤ 1/32S | ≤ 1/32S |
| Erythromycin | macrolides | 4R | 2R |
| Vancomycin | glycopeptides | ¼S | ⅛S |

The following patents, applications and publications as listed below and throughout this document are hereby incorporated by reference in their entirety herein.

### REFERENCES

1. Barrasa-Villar, J. I.; Aibar-Remón, C.; Prieto-Andrés, P.; Mareca-Doñate, R.; Moliner-Lahoz, J., Impact on Morbidity, Mortality, and Length of Stay of Hospital-Acquired Infections by Resistant Microorganisms. Clin Infect Dis 2017, 65 (4), 644-652.
2. Cosgrove, S. E.; Sakoulas, G.; Perencevich, E. N.; Schwaber, M. J.; Karchmer, A. W.; Carmeli, Y., Comparison of mortality associated with methicillin-resistant and methicillin-susceptible Staphylococcus aureus bacteremia: a meta-analysis. Clin Infect Dis 2003, 36 (1), 53-9.
3. Giske, C. G.; Monnet, D. L.; Cars, O.; Carmeli, Y., Clinical and economic impact of common multidrug-resistant gram-negative bacilli. Antimicrob Agents Chemother 2008, 52 (3), 813-21.
4. Karam, G.; Chastre, J.; Wilcox, M. H.; Vincent, J.-L., Antibiotic strategies in the era of multidrug resistance. Critical Care 2016, 20, 136.
5. Prevention, C. f. D. C. a. Antibiotic Resistance Threats in the United States, 2019. (accessed June 18).
6. Thorpe, K. E. J., P.; Johnston K. J., Antibiotic-Resistant Infection Treatment Costs Have Doubled Since 2002, Now Exceeding $2 Billion Annually. Health Affairs 2018, 37 (4), 662-669.
7. Organization, W. H., Antimicrobial resistance: global report on surveillance. World Health Organization: 2014.
8. Musicha, P.; Cornick, J. E.; Bar-Zeev, N.; French, N.; Masesa, C.; Denis, B.; Kennedy, N.; Mallewa, J.; Gordon, M. A.; Msefula, C. L.; Heyderman, R. S.; Everett, D. B.; Feasey, N. A., Trends in antimicrobial resistance in bloodstream infection isolates at a large urban hospital in Malawi (1998-2016): a surveillance study. The Lancet. Infectious diseases 2017, 17 (10), 1042-1052.
9. Santella, B.; Folliero, V.; Pirofalo, G. M.; Serretiello, E.; Zannella, C.; Moccia, G.; Santoro, E.; Sanna, G.; Motta, O.; De Caro, F., Sepsis—A Retrospective Cohort Study of Bloodstream Infections. Antibiotics 2020, 9 (12), 851.
10. Kollef, M. H., Broad-spectrum antimicrobials and the treatment of serious bacterial infections: getting it right up front. Clin Infect Dis 2008, 47 Suppl 1, S3-13.
11. Micek, S. T.; Hampton, N.; Kollef, M., Risk Factors and Outcomes for Ineffective Empiric Treatment of Sepsis Caused by Gram-Negative Pathogens: Stratification by Onset of Infection. Antimicrob Agents Chemother 2018, 62 (1).
12. Rhee, C.; Kadri, S. S.; Dekker, J. P.; Danner, R. L.; Chen, H.-C.; Fram, D.; Zhang, F.; Wang, R.; Klompas, M., Prevalence of antibiotic-resistant pathogens in culture-proven sepsis and outcomes associated with inadequate and broad-spectrum empiric antibiotic use. JAMA Network Open 2020, 3 (4), e202899-e202899.
13. Kang, C.-I.; Kim, S.-H.; Park, W. B.; Lee, K.-D.; Kim, H.-B.; Kim, E.-C.; Oh, M.-d.; Choe, K.-W., Bloodstream Infections Caused by Antibiotic-Resistant Gram-Negative Bacilli: Risk Factors for Mortality and Impact of Inappropriate Initial Antimicrobial Therapy on Outcome. Antimicrobial Agents and Chemotherapy 2005, 49 (2), 760-766.
14. Chen, J.-R.; Lee, S.-Y.; Yang, B.-H.; Lu, J.-J., Rapid identification and susceptibility testing using the VITEK 2 system using culture fluids from positive BacT/ALERT blood cultures. Journal of microbiology, immunology, and infection 2008, 41 (3), 259-264.
15. Ling, T. K.; Liu, Z.; Cheng, A. F., Evaluation of the VITEK 2 system for rapid direct identification and susceptibility testing of gram-negative bacilli from positive blood cultures. Journal of clinical microbiology 2003, 41 (10), 4705-4707.
16. Höring, S.; Massarani, A. S.; Löffler, B.; Rödel, J., Rapid antibiotic susceptibility testing in blood culture diagnostics performed by direct inoculation using the VITEK®-2 and BD Phoenix™ platforms. European Journal of Clinical Microbiology & Infectious Diseases 2019, 38 (3), 471-478.
17. Florio, W.; Barnini, S.; Morici, P.; Lupetti, A., Direct inoculation of positive blood cultures using the Phoenix system for antimicrobial susceptibility testing of both Gram-positive and Gram-negative bacteria. J Med Microbiol 2015, 64 (Pt 5), 582-585.
18. Beuving, J.; van der Donk, C. F. M.; Linssen, C. F. M.; Wolffs, P. F. G.; Verbon, A., Evaluation of direct inoculation of the BD PHOENIX system from positive BACTEC blood cultures for both Gram-positive cocci and Gram-negative rods. BMC Microbiology 2011, 11 (1), 156.
19. Lupetti, A.; Barnini, S.; Castagna, B.; Nibbering, P.; Campa, M., Rapid identification and antimicrobial susceptibility testing of Gram-positive cocci in blood cultures by direct inoculation into the BD Phoenix system. Clinical microbiology and infection 2010, 16 (7), 986-991.
20. Walsh, J. D.; Hyman, J. M.; Borzhemskaya, L.; Bowen, A.; Mckellar, C.; Ullery, M.; Mathias, E.; Ronsick, C.; Link, J.; Wilson, M.; Clay, B.; Robinson, R.; Thorpe, T.; van Belkum, A.; Dunne, W. M., Rapid Intrinsic Fluorescence Method for Direct Identification of Pathogens in Blood Cultures. mBio 2013, 4 (6), e00865-13.
21. Dorn, G. L.; Burson, G. G.; Haynes, J. R., Blood culture technique based on centrifugation: clinical evaluation. Journal of clinical microbiology 1976, 3 (3), 258-263.
22. Dorn, G. L.; Haynes, J. R.; Burson, G. G., Blood culture technique based on centrifugation: developmental phase. Journal of Clinical Microbiology 1976, 3 (3), 251-257.
23 Mortimer, M.; Petersen, E. J.; Buchholz, B. A.; Holden, P. A., Separation of Bacteria, Protozoa and Carbon Nanotubes by Density Gradient Centrifugation. Nanomaterials (Basel, Switzerland) 2016, 6 (10).
24. Pillai, S. D.; Josephson, K. L.; Bailey, R. L.; Gerba, C. P.; Pepper, I. L., Rapid method for processing soil samples for polymerase chain reaction amplification of specific gene sequences. Appl Environ Microbiol 1991, 57 (8), 2283-2286.
25. Assunção, P.; Antunes, N. T.; Rosales, R. S.; Poveda, C.; De La Fe, C.; Poveda, J. B.; Davey, H. M., Application of flow cytometry for the determination of minimal inhibitory concentration of several antibacterial agents on Mycoplasma hyopneumoniae. J Appl Microbiol 2007, 102 (4), 1132-7.
26. Costa-de-Oliveira, S.; Teixeira-Santos, R.; Silva, A. P.; Pinho, E.; Mergulhão, P.; Silva-Dias, A.; Marques, N.; Martins-Oliveira, I.; Rodrigues, A. G.; Paiva, J. A.; Cantón, R.; Pina-Vaz, C., Potential Impact of Flow Cytometry Antimicrobial Susceptibility Testing on the Clinical Management of Gram-Negative Bacteremia Using the FASTinov® Kit. Frontiers in Microbiology 2017, 8 (2455).
27. Ordóñez, J. V.; Wehman, N. M., Rapid flow cytometric antibiotic susceptibility assay for Staphylococcus aureus. Cytometry: The Journal of the International Society for Analytical Cytology 1993, 14 (7), 811-818.
28 Broeren, M. A. C.; Maas, Y.; Retera, E.; Arents, N. L. A., Antimicrobial susceptibility testing in 90 min by bacterial cell count monitoring. Clin Microbiol Infect 2013, 19 (3), 286-291.
29 Faria-Ramos, I.; Espinar, M.; Rocha, R.; Santos-Antunes, J.; Rodrigues, A. G.; Cantón, R.; Pina-Vaz, C., A novel flow cytometric assay for rapid detection of extended-spectrum beta-lactamases. Clinical Microbiology and Infection 2013, 19 (1), E8-E15.
30 Gant, V.; Warnes, G.; Phillips, I.; Savidge, G., The application of flow cytometry to the study of bacterial responses to antibiotics. Journal of Medical Microbiology 1993, 39 (2), 147-154.
31. Huang, T.-H.; Ning, X.; Wang, X.; Murthy, N.; Tzeng, Y.-L.; Dickson, R. M., Rapid Cytometric Antibiotic Susceptibility Testing Utilizing Adaptive Multidimensional Statistical Metrics. Analytical Chemistry 2015, 87 (3), 1941-1949.
32. Huang, T.-H.; Tzeng, Y.-L.; Dickson, R. M., FAST: Rapid Determinations of Antibiotic Susceptibility Phenotypes using Label-Free Cytometry. Cytometry A 2017, Submitted.
33 Clinical and Laboratory Standards Institue (CLSI), Performance Standards for Antimicrobial Susceptibility Testing; Twenty-Fourth Informational Supplement. Clinical and Laboratory Standards Institute: Wayne, P A, 2014; Vol. M100-S24.
34. Chirife, J.; Herszage, L.; Joseph, A.; Kohn, E. S., In vitro study of bacterial growth inhibition in concentrated sugar solutions: microbiological basis for the use of sugar in treating infected wounds. Antimicrobial agents and chemotherapy 1983, 23 (5), 766-77.
35. Shrestha, N. K.; Scalera, N. M.; Wilson, D. A.; Procop, G. W., Rapid differentiation of methicillin-resistant and methicillin-susceptible Staphylococcus aureus by flow cytometry after brief antibiotic exposure. Journal of clinical microbiology 2011, 49 (6), 2116-2120.
36. Suller, M.; Lloyd, D., Fluorescence monitoring of antibiotic-induced bacterial damage using flow cytometry. Cytometry: The Journal of the International Society for Analytical Cytology 1999, 35 (3), 235-241.
37. Mortimer, F. C.; Mason, D. J.; Gant, V. A., Flow cytometric monitoring of antibiotic-induced injury in Escherichia coli using cell-impermeant fluorescent probes. Antimicrobial agents and chemotherapy 2000, 44 (3), 676-681.
38 Gauthier, C.; St-Pierre, Y.; Villemur, R., Rapid antimicrobial susceptibility testing of urinary tract isolates and samples by flow cytometry. Journal of medical microbiology 2002, 51 (3), 192-200.
39 Bär, W.; Bade-Schumann, U.; Krebs, A.; Cromme, L., Rapid method for detection of minimal bactericidal concentration of antibiotics. Journal of Microbiological Methods 2009, 77 (1), 85-9.

1. A method of obtaining an enriched population of bacterial cells from a biological sample, the method comprising the steps of:
a. Providing a tube (1) comprising a separating material (2), wherein a proximal end (3) of the tube is comprised of a stopper (4) which allows entry of a needle (5), and wherein the separating material is in the proximal end of the tube so that the separating material is in contact with the proximal end of the tube;
b. Inserting the biological sample (6) into the tube, wherein the biological sample is layered on the distal side of the separating material, so that the separating material is not in contact with the proximal end of the tube;
c. Centrifuging the tube for a sufficient time to cause bacterial cells to migrate into the separating material, wherein 50% or more of mammalian cells are excluded from the separating material; and
d. Withdrawing a sample from the separating material, wherein said sample comprises an enriched population of biological cells.

2. The method of claim 1, wherein the biological sample is a bodily fluid or a culture of a patient sample.

3. (canceled)

4. (canceled)

5. The method of claim 1, wherein the tube is a vacutainer tube.

6. The method of claim 5, wherein the vacutainer tube is inverted, so that the proximal end which allows entry of the needle is pointed downwards.

7. The method of claim 1, wherein the separating material forms a step or continuous density gradient.

8. The method of claim 1, wherein the separating material comprises a sugar, carbohydrate, small molecule, oligomer, or polymer dissolved or suspended in buffer, biological media or water.

9. The method of claim 8, wherein the separating material comprises sucrose.

10. The method of claim 1, wherein the tube is centrifuged long enough to concentrate bacteria in the separating material.

11. (canceled)

12. (canceled)

13. (canceled)

14. (canceled)

15. (canceled)

16. (canceled)

17. The method of claim 1, wherein the biological sample is treated with a detergent which lyses mammalian cells before or after the biological sample is inserted into the tube.

18. The method of claim 17, wherein the detergent does not lyse bacterial cells, or lyses bacterial cells with less efficiency than mammalian cells.

19. The method of claim 17, wherein the detergent is saponin or sodium dodecylsulfate (SDS).

20. The method of claim 1, wherein a needle is used to insert the biological sample into the tube.

21. The method of claim 20, wherein said needle is attached to a syringe.

22. The method of claim 21, wherein said syringe and needle are separated by a stopcock or automated pump and switch.

23. The method of claim 22, wherein said stopcock is a three-way stopcock.

24. The method of claim 1, wherein after step d), the sample is analyzed or further processed.

25. The method of claim 24, wherein the sample is subjected to an antimicrobial susceptibility test.

26. The method of claim 1, wherein after step b), but before step c), air is removed from the region distal to the layer comprising the biological sample.

27. A tube for separating bacteria from mammalian cells, wherein the tube comprises a separating material, wherein a proximal end of the tube is comprised of a material which allows entry of a needle, and wherein the separating material is in the proximal end of the tube so that the separating material is in contact with the proximal end of the tube.

28. (canceled)

29. (canceled)

30. (canceled)

31. (canceled)

32. (canceled)

33. A kit for obtaining a bacterial sample, the kit comprising a tube for separating bacteria from mammalian cells, wherein the tube comprises a separating material, wherein a proximal end of the tube is comprised of a material which allows entry of a needle, and wherein the separating material is in the proximal end of the tube so that the separating material is in contact with the proximal end of the tube.

34. (canceled)

35. (canceled)

36. (canceled)

37. (canceled)

38. (canceled)

39. (canceled)

40. (canceled)
